(19) United States
(12) Patent Application Publication
(10) Pub. No.: US 2004/0171613 A1
(43) Pub. Date: Sep. 2, 2004

(54) Therapeutic agent for non-immediate type allergic diseases

(75) Inventors: Hiroyuki Iwamura, Osaka (JP); Yoshifumi Ueda, Osaka (JP)

Correspondence Address: FOLEY AND LARDNER, SUITE 500, 3000 K STREET NW, WASHINGTON, DC, 20007, US

(73) Assignee: JAPAN TOBACCO INC.

(21) Appl. No.: 10/734,577

(22) Filed: Dec. 15, 2003

Related U.S. Application Data
(63) Continuation-in-part of application No. 10/375,057, filed on Feb. 28, 2003.
Application No. 10/375,057 is a continuation of application No. PCT/JP02/13806, filed on Dec. 27, 2002.

(30) Foreign Application Priority Data
Dec. 27, 2001 (JP) 396981/2001

(51) Int. Cl. A61K 31/541; A61K 31/5377; A61K 31/4709; A61K 31/4706

(52) U.S. Cl. 514/227.8; 514/312; 514/231.2

(57) ABSTRACT

An objective of the present invention is to provide novel therapeutic agents for non-immediate-type allergic diseases that comprise, as an active ingredient, a cannabinoid receptor modulator, particularly that selectively acts on peripheral cell type cannabinoid receptors (CB2), and more particularly an inverse agonist.

The present invention provides therapeutic agents for non-immediate-type allergic diseases which comprises a cannabinoid receptor modulator, particularly an inverse agonist that selectively acts on peripheral cell type cannabinoid receptors, specifically N-(benzo[1,3]dioxol-5-yl methyl)-7-methoxy-2-oxo-8-pentyloxy-1,2-dihydroquinoline-3-carboxamide or such, or pharmaceutically acceptable salt thereof. The therapeutic agents of the present invention are effective, for example, against intractable allergic diseases, such as asthma and atopic dermatitis.

### FIELD OF THE INVENTION

The present invention relates to a novel use of cannabinoid receptor-inverse agonists. More specifically, the invention relates to a use of an inverse agonist selectively acting on cannabinoid receptors, particularly peripheral cell type receptors (also referred to as CB2), as a therapeutic agent for non-immediate type allergic diseases and a method of treating non-immediate type allergic diseases using the inverse agonist.

### BACKGROUND OF THE INVENTION

[Indian hemp and cannabinoid]

Hemp has been used as a drug for analgesic action, antipyretic action, hypnotic action and the like from ancient times. In Japan, from 1886 to 1951, the Japanese Pharmacopoeia describes hemp as Indian hemp for use as analgesics and anesthetic agent. In USA, from 1850 to 1942, the Pharmacopoeia describes alcohol solution of hemp as a pharmaceutical agent for rheumatoid arthritis, asthma, tonsillitis, etc.

On the other hand, both hemp and Δ9-tetrahydrocannabiol (THC), which is considered to be the main ingredient of hemp for expressing psychological actions, have been known to induce visual or hearing disorder, cognition disorder of time and space, suggestibility elevation, reduction of thinking activity and initiative, and memory disorders, and thereby cause distinct change in the psychological function. In addition to these actions, markedly divers pharmacological actions are reported including, for example, motor ataxia, over sensitivity, hypothermia, respiratory suppression, tachycardia, catalepsy-triggering action, hypertension, vasodilating action, immunosuppressive action and hydrodipsia. Currently, its use is under regulation.

A series of hallucinogens comprised in hemp are collectively referred to as cannabinoid. To date, 60 types or more of cannabinoids, including THC have been found.

Various artificial ligands stronger than naturally occurring cannabinoids have been developed. The receptors thereof have also been screened. Consequently, in 1988, a cannabinoid receptor was shown to exist as a membrane component of rat brain and, subsequently, in 1991, human cDNA encoding the receptor was cloned. Furthermore, a protein with 44% homology to the receptor encoded by the human cDNA was found in human promyelocytic leukemia cell HL60. Thereafter, it was confirmed that the protein was distributed in peripheral tissues such as spleen. In 1993, Munro et al. proposed to call the receptors in brain “CB1” and those found in peripheral tissues “CB2”. Currently, these designated terms are generally used.

In addition to brain, although with far lower levels of expression, CB1 has been detected in many tissues including human testis, human prostate, ovary, uterus, bone marrow, thymus, tonsil, pituitary gland, adrenal gland, heart, lung, stomach, large intestine, bile duct, leukocyte, etc. In contrast, CB2 does not exist in the brain of rat but was observed in monocytes in the spleen marginal zone. In human spleen, leukocyte, tonsil, thymus and pancreas, CB2 exists at a far higher level than the CB1.

The concrete substances of the two subtypes (CB1 and CB2) of the receptor were confirmed. Furthermore, the existence of endogenous ligands of the receptor, such as anandamide, 2-arachidonoylglycerol (2-AG) and 2-arachidonoylglycerol ether (2-AG-E; reduced 2-AG that is suggested to have similar function to 2-AG) was confirmed. Moreover, the physiological roles of the receptors have been examined and various findings are being made. Reported findings include the finding that CB2 suppresses the proliferation of T cell and B cell to induce apoptosis and exert immunosuppressive action, the finding that no central action by cannabinoid treatment is exhibited in CB1-defective knockout mice, and the finding that no suppression of helper T cell due to cannabinoid is observed in CB2-defective knockout mice.

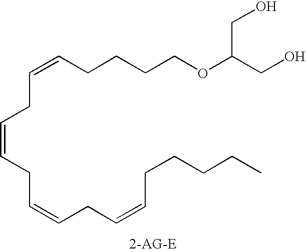

Currently, on the basis of these findings, difference in distribution and function between CB1 and CB2 has been examined to apply agonists, antagonists or inverse agonists respectively specific to these subtypes to pharmaceutical products. In relation with CB1, for example, Parkinson's disease, Alzheimer's disease, memory disorders, senile dementia, multiple sclerosis, appetite loss and pain have been targeted for creating pharmaceutical agents. In relation with CB2, for example, immune disease, rheumatoid arthritis and inflammation have been targeted for creating pharmaceutical agents. Particularly, pharmaceutical agents selectively exerting actions on CB2, i.e., peripheral cell type (also referred to as peripheral type or periphery) cannabinoid receptor selective modulators are expected as safe pharmaceuticals with no central action. Cannabinoids show central action on CB1 at a very low concentration. Thus, a CB2-selective modulator with lower CB1 action is desired.

Currently known ligands for non-selective cannabinoid receptor include, for example, Δ9-THC, CP55940, WIN55212-2, HU-243 and HU-210; and known CB1-selective ligands include SR141716A, LY320135, arachidonoyl-2′-chloroethylamide and CP56667; and known CB2-selective ligands include SR144528, AM630, HU-308, JWH-051 and L-768242 (see, for example, non-patent references 1 and 2).

[Allergy]

Herein, allergic diseases, particularly allergic dermatitis and allergic asthma are described.

Allergy is recognized as a hypersensitive biological reaction based on antigen-antibody reaction, which is different from general inflammatory reaction that involves characteristic accumulation of monocytes, macrophages and neutrophils. Eosinophils, basophils and mast cells are largely involved in allergic reaction.

Generally, allergic reaction is now classified in four types. However, these four reactions do not independently occur in the body and in some cases, a few of these reactions occur simultaneously.

When an antigen (allergen) invades into the body, first, it is incorporated into an antigen presenting cell such as macrophages.

The antigen presenting cell transmits the information of the incorporated antigen to T cells. Then, the T cells order B cells to produce antigen-specific IgE antibody. The IgE antibody binds to a mast cells, so that the mast cells are sensitized.

When the antigen invades again into the body and binds to the IgE antibody on the mast cells, various chemical mediators, such as histamine, eosinophil chemotactic factor and leukotrienes, and cytokines, such as interleukin, are released from the mast cells.

When such chemical mediators act on the bronchi, the smooth muscle of the bronchi constricts to cause mucosal swelling and sputum secretion to narrow its airway, finally causing asthma attack. When such chemical mediators exert its action on skin, inflammation, swelling and itching occur to cause dermal diseases such as urticaria. Furthermore, when such chemical mediators act on nasal mucus, vascular permeability is increased and exudate draws out of blood which may cause swelling of nasal mucosa leading to nasal occlusion or allergic rhinitis involving sternutation and discharge of a large volume of pituita via nervous irritation. When the reaction occurs in digestive tract, intestinal smooth muscle constricts to abnormally increase intestinal motion (vermiculation), causing digestive allergies such as abdominal pain, vomiting and diarrhea.

This reaction occurs within 30 minutes after antigen invasion. Therefore, the reaction is referred to as immediate type allergic reaction or Type I allergic reaction. Generally, the immediate type allergic reaction disappears in about one hour. Typical diseases of the immediate type allergic reaction include anaphylaxis, allergic rhinitis, pollenosis, urticaria and allergic gastrointestinal diseases.

However, several hours to several days later, eosinophils comprising highly toxic chemical mediators are gathered around the site of allergic reaction due to eosinophilic chemotactic factors and cytokines that were released from mast cells. The gathered eosinophils release chemical mediators to trigger tissue damages. This reaction is referred to as “late phase allergic reactions”. When this reaction occurs in bronchi, the mucoepithelium detaches and antigens more readily invade in the bronchi, leading to prolonged allergic reaction and elevation of the hypersensitivity of airway, making asthma intractable. This is referred to as late asthmatic response. For example, such late phase response mainly occur after 4 to 8 hours in the case of asthma and mainly occur after 12 to 48 hours in the case of atopic dermatitis.

Type II allergic reaction is also referred to as cytolysis type allergic reaction, wherein complements act on antigen bound IgM or IgG antibody to open holes through the cell membrane to lyse cells. In addition, a reaction occurs wherein macrophages or killer cells act on antibody bound cells and release damaging substances to damage the cells or tissue. Typical diseases of the type II allergic reaction include hemolytic anemia, idiopathic thrombocytopenic purpura, myasthenia gravis and Goodpasture syndrome.

In a type III allergic reaction, phagocytes get incapable to process antigen-antibody complexes composed of an antigen and an antibody (IgG antibody) bound together, and the antigen-antibody complexes deposit on tissues. Then, complement, macrophages and neutrophils accumulate on the deposited site to cause inflammation and damage the tissues. Typical diseases of the type III allergic reaction include acute glomerulonephritis induced by hemolytic streptococcus, rheumatoidarthritis, collagen disease, serum sickness, viral hepatitis and allergic alveolitis.

Type IV allergic reaction is different from the type I to III reactions in that no antibody is involved in the reaction. Provided that sensitization with an antigen is established, when the antigen infiltrates again, T cells release cytokines to migrate immune cells such as lymphocytes, neutrophils and macrophages, and destroy the antigen, but at the same time induces inflammation to cause tissue damages. When the infiltrating antigen is a cell, killer T cell damages the cell (antigen). The reaction is generally completed in one to 2 days, and is also referred to as “delayed-type allergic reaction”. Type IV allergic reactions include tuberculin reaction, tuberculosis lesion, post-organ grafting rejections and dermatitis, such as rash against Japanese lacquer (urushi) and rash against cosmetics.

Most of the acute symptoms of common allergic diseases, such as allergic asthma, atopic dermatitis, allergic rhinitis and allergic conjunctivitis, have been classified as immediate type response. In recent years, however, it has been recognized that allergic asthma essentially is not a transient immediate type hypersensitivity but a chronic inflammation.

“Allergic asthma” induced by allergen and non-allergic asthma induced not by a specific allergen but by coldness, athletic motion and the like are known as asthma.

“Asthma”, namely “bronchial asthma” has been characterized by its reversible airstream restriction (airway occlusion) and airway hypersensitivity. However, airways suffering from asthma were clarified to involve the occurrence of chronic inflammation characterized by detachment of airway epithelium, fibrosis of airway just below the basement membrane (hypertrophy of the basement membrane) and eosinophil accumulation. Currently, asthma is therefore recognized as a chronic inflammatory disease. Many inflammatory cells such as eosinophils, T cells and mast cells are suggested to be involved in airway inflammation. It is considered that the involvement of mast cells, the involvement of eosinophils, and the involvement of eosinophils and CD4-positive helper T cells are important for immediate type response, late phase response and delayed type response, respectively.

In relation to anti-asthma agents, therapeutic treatment of reversible airway occlusion mainly with bronchial dilator has been replaced with therapeutic treatment of chronic inflammation mainly with anti-inflammatory agent. For therapeutic treatment of asthma attacks, depending on the symptom, short-term acting β₂ stimulators, short-term acting theophyllines, anti-choline agents for inhalation, steroidal agents for injections or oral dosing are used. On the other hand, for long-term control, in addition to steroidal agents for inhalation and oral dosing, sustained-release type theophyllines and long-term acting β₂ stimulators, anti-allergic agents (mediator release-inhibitors, histamine H₁ antagonists, leukotriene antagonists, thromboxane A2 inhibitors and antagonists, and Th2 cytokine inhibitors) are used. However, side effects such as suppression of adrenal function are known for the steroidal agents and some symptoms show little effect (resistance) to steroids and leukotriene antagonists. Therefore, additional anti-asthmatic agents are desired in the art.

Atopic asthma and atopic dermatitis are symptoms of allergic diseases with family history or anamnesis. Asthma and dermatitis of atopic type are frequently observed in children. Therefore, a therapeutic agent with particularly less side effects is desired.

In definition, “Atopic dermatitis” is a disease involving eczema with itching as the main lesion which repeats exacerbation and remission. Many of the patients of atopic dermatitis have atopic predisposition. Atopic predisposition is: (1) having family history or anamnesis (any disease or plural diseases of bronchial asthma, allergic rhinitis, conjunctivitis and atopic dermatitis), or (2) a predisposition readily generating IgE antibody”, and thus, atopic dermatitis is discriminated from other inflammatory dermatitis.

Symptoms of atopic dermatitis include hypersensitivity and dryness of skin. Characteristic exanthema of atopic dermatitis (erythematosus, papule, incrustation, lichen lesion, prurigo, etc.) progress in chronic and recurrent course. Further, the symptoms induce complications such as Kaposi's sarcoma varicelliform eruption, viral infections (infection with herpes simplex virus and the like), impetigo and infectious molluscum (cataract, retinodialysis, etc.)

In addition to immediate type and late phase allergic reactions via IgE and mast cells, delayed type allergic reaction via Langerhans cells and T cells are also considered to be involved in the lesion of atopic dermatitis.

For therapeutic treatment of atopic dermatitis, pharmaceutical therapy is used depending on the symptom, together with the elimination of causes or exacerbating factors, such as foods or mite, and skincare (keeping the skin clean and using moisturizing agents to prevent skin dryness).

Anti-histamine agents are used for itching. However, the effect is not so distinct as in the case of urticaria.

For inflammation, principally, external steroidal application agents such as predonisolone and betamethasone valerate are used. Oral dosage forms of anti-histamine agents or anti-allergy agents are used in a supplementary manner. However, dermatitis is believed to be difficult to control with these agents alone. Generally, atopic dermatitis is intractable and many patients reject steroidal agents due to their side effect. Thus, a new pharmaceutical agent is required in the art. In recent years, an immunosuppressor, tacrolimus ointment, has been used and shows some effect. However, due to its side effects, its use is restricted. For therapeutic treatment of symptoms on skin lesions with severe damage which makes external application difficult, symptoms occurring at sensitive sites with originally thin epithelium such as face and mucus, and diseases in the inner layer of epithelium or over a wide area of the body, development of oral agents that are safe and ready to handle is also desired in the art.

Unexamined Published Japanese Patent Application No. (JP-A) 2000-256323 (WO 00/40562), an application filed by the present applicant teaches 2-oxoquinoline compound represented by the following general formula as a cannabinoid receptor-modulator.

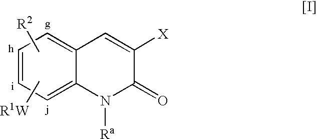

(In the formula, the symbols are as described therein.)

Furthermore, JP-A 2000-256323 describes N-(benzo[1,3]dioxol-5-ylmethyl)-7-methoxy-2-oxo-8-pentyloxy-1,2-dihydroquinoline-3-carboxamide (hereinafter referred to as compound A) as an example of the 2-oxoquinoline compound.

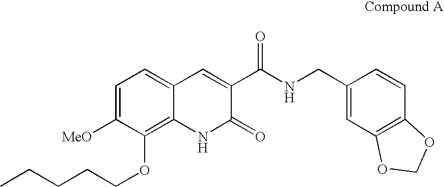

Further, the publication describes, in relation to the use of the cannabinoid receptor-modulators, that “based on the discovery of a peripheral cell type receptor, for example, a receptor on macrophage (see non-patent reference 6), agonists of the peripheral cell type receptor are being developed, which exert anti-inflammatory and anti-allergy actions via the regulation of immune reaction and which originally has immunoregulatory action”, “the development of a peripheral cell type receptor-selective modulator is particularly desired because a pharmaceutical agent that selectively acts on peripheral cell type cannabinoid receptor may serve as safe pharmaceuticals that does not exerts side effects, central actions such as hypothermia and catalepsy”, and “are useful as cannabinoid receptor (particularly peripheral type cannabinoid receptor)-modulator, immunomodulators, therapeutic agents of autoimmune diseases, anti-inflammatory agents and anti-allergic agents”.

Additionally, the publication describes pharmacological studies including selective binding to peripheral cell type cannabinoid receptor (CB2), a carrageenan-induced paw edema model and a suppression of inflammation and bleeding of pancreas in rat taurocol acid-induced pancreatitis model (see patent reference 1).

Although JP-A 2000-256323 specifically discloses anti-inflammatory action of the compound, it fails to disclose specific test examples and data on allergic diseases. Nor does the publication specifically teach about particular allergic diseases such as atopic dermatitis and allergic asthma.

Meanwhile, a related-art reference describes that compound A described above and SR144528 described below are CB2-selective ligands and that they function as CB2 inverse agonists. More specifically, the related-art reference describes that compound A and SR144528 elevate the production of cyclic adenosine-phosphate (cAMP) stimulated with forskolin (an adenylate cyclase activating agent) in CB2 expressing CHO cells, i.e., describes that compound A and SR144528 function as CB2 inverse agonists. This reference concurrently describes about a general finding that THC reduced cAMP production in the test (see non-patent reference 3).

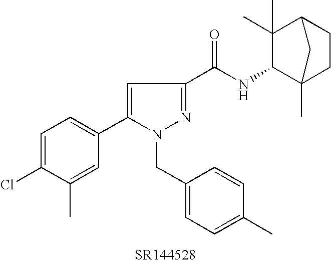

Some known patent publications describe about anti-allergic effect of cannabinoid modulators.

JP-A Sho 52-113976 (U.S. Pat. No. 4,179,517) describes the effect of THC derivative on the prevention of asthma attack and describes asthma, allergy and the like as the indication (see patent reference 2).

Published Japanese translation of International Publication No. (JP-T) 2002-511411 (WO 99/52524) describes that cannabinoids, such as cannabidiol, can be used for therapeutic treatment of inflammatory diseases, such as asthma. However, it cites a reference describing cannabidiol as not binding to CB1 or CB2 (see patent reference 3).

WO 01/64212 describes that cannabinoid modulators, preferably CB1 agonist, can be used for therapeutic treatment of muscle diseases, for example, asthma, bronchitis, etc. (see patent reference 4).

WO 01/95899 describes anti-inflammatory action of cannabidiol derivative on arachidonate-induced ear edema (see patent reference 5).

WO 01/89589 describes a method for ameliorating cough by local administration of cannabinoid to regulate CB1 receptor existing in peripheral cells (see patent reference 6).

WO 00/16756 discloses a cannabinoid modulator, and dermal diseases (atopic dermatitis, etc.), respiratory diseases (asthma, etc.), allergic rhinitis and the like as the indication. WO 00/16756 also describes that the compound is nonetheless CB1-selective and regulates CB1 receptor existing in peripheral cells (see patent reference 7).

JP-T Hei 8-504195 (WO 94/12466) describes that a ligand for the cannabinoid receptor exerts anti-inflammatory activity, anti-asthmatic activity and so on (see patent reference 8).

JP-A Hei 6-73014 (U.S. Pat. No. 5,624,941) and JP-A Hei 7-324076 (U.S. Pat. No. 5,462, 960) describe that ligands for the cannabinoid receptor can be used for therapeutic treatment of thymus disorders, asthma, immunoregulation, and the like (see patent references 9 and 10).

WO 01/98289 describes that compounds of Δ6-tetrahydrocannabiol type can be used for therapeutic treatment of inflammation, lung diseases such as asthma and chronic occlusive lung disease, autoimmune disease and the like. It further describes that the action is exerted via the inhibition of prostaglandin synthesis, inhibition of tumor necrosis factor production, cyclooxygenase inhibition and inhibition of nitric oxide production, in addition to blocking of N-methyl-D-aspartic acid receptor and anti-oxidative activity (see patent reference 11).

WO 02/26702 describes that cannabinoid receptor-modulator, particularly agonists, are effective for asthma, allergy, dermal diseases and the like (see patent reference 12).

WO 01/87297 describes that CB1 modulator can be used for therapeutic treatment of dermal necrosis such as psoriasis (see patent reference 13).

WO 02/42248 describes that cannabinoid receptor-binding agents, particularly CB1 agonists, can be used for asthma, rhinitis and inflammatory dermal diseases (see patent reference 14).

WO 02/47691 describes that cannabinoid receptor agonists can be used for therapeutic treatment of inflammation and the like (see patent reference 15).

However, hitherto, none of these compounds have been experimentally proven to possess therapeutic effect on allergic diseases, particularly atopic dermatitis.

Further, some publications describe about the pharmacological action of CB2 selective cannabinoid modulator.

JP-T Hei 11-500411 (WO 96/18391) describes that CB2 modulators can be used for therapeutic treatment of immune system disorders, chronic respiratory disorders (asthma, etc.) and the like, and further describes that CB2 expression was observed in mast cells and non-immune cells (for example, cerebellar granule, cerebellum and heart) (see patent reference 16).

JP-T Hei 11-501615 (WO 96/18600) describes that CB2 modulators can be used for therapeutic treatment of autoimmune diseases, chronic inflammation, respiratory disorders (asthma, etc.) and the like (see patent reference 17).

JP-T Hei 10-508870 (WO 96/25397) describes that CB2 modulators can be used for therapeutic treatment of lung disorders (asthma, chronic bronchitis, etc.), allergic reactions (rhinitis, contact dermatitis, conjunctivitis, etc.) and immune system disorders (see patent reference 18).

JP-T Hei 11-507937 (U.S. Pat. No. 6,013,648) discloses an agent acting on CB2 and describes autoimmune diseases, infectious diseases and allergic diseases (specifically, acute hypersensitivity and asthma) as the indication thereof. This agent is described as selectively acting on CB2, however suppresses cAMP production stimulated by forskolin (see patent reference 19).

JP-T 2000-502080 (U.S. Pat. No. 5,925,768) describes compounds that have affinity for the CB2 receptor and describes immune diseases, for example, allergic diseases (immediate type hypersensitivity and asthma) as the indication. The reference, however, describes that the compounds are CB2 receptor antagonists (see patent reference 20).

JP-T 2001-508799 (WO 98/31227) describes that CB2 modulators, particularly antagonists can be used for therapeutic treatment of immune diseases, inflammation and the like (see patent reference 21).

JP-T 2001-516361 (WO 98/41519) describes that CB2 modulators, particularly agonists can be used for therapeutic treatment of immune diseases, inflammation and the like (see patent reference 22).

JP-T 2001-515470 (U.S. Pat. No. 6,262,112) describes that cannabinoid agonists, particularly CB1 agonists are effective for therapeutic treatment of allergic diseases, asthma, inflammatory dermal diseases and/or dermal diseases ascribed to immunological causes. Furthermore, JP-T 2001-515470 describes that some of the compounds are effective for CB2 (see patent reference 23).

WO 99/57107 describes that CB2-selective modulators can be used for anti-inflammation and immunoregulation (see patent reference 24).

JP-T 2002-523395 (WO 00/10967) and JP-T 2002-523396 (WO 00/10968) teach that both CB1 agonists and CB2 agonists can be used for therapeutic treatment of dermal diseases and the like (see patent references 25 and 26).

JP-T 2002-539246 (WO 00/56303) teaches that CB2-selective agonists can be used for therapeutic treatment of immune diseases (see patent reference 27).

WO 01/4083 describes that CB2-selective modulators, particularly agonists can be used for therapeutic treatment of inflammation, immunological diseases, for example, atopic dermatitis, allergic dermatitis, asthma, etc. However, the publication describes that the compounds suppress cAMP-increase (see patent reference 28).

WO 01/19807 describes that CB2-selective modulators, particularly agonists have anti-inflammatory and immunosuppressive actions and describes test results of experiments on sheep erythrocyte-induced delayed type hypersensitivity model. However, the publication describes that the compounds suppress cAMP-increase (see patent reference 29).

WO 01/29007 describes that cannabinoid modulators can be used for anti-inflammation, regulation of immune system and so on. The publication describes that some of the compounds are antagonists and others agonists, and further describes CB2-selective modulators based on binding assay results (see patent reference 30).

WO 01/28497 describes that CB2-selective modulators, particularly agonists have anti-inflammatory actions and the like (see patent reference 31).

WO 01/32169 describes that CB2-selective agonists can be used for anti-inflammation, and therapeutic treatment of autoimmune diseases and the like (see patent reference 32).

WO 01/28329 describes that CB2-selective substances can be used for anti-inflammation, and therapeutic treatment of autoimmune diseases and the like (see patent reference 33).

WO 01/28557 describes that cannabinoid receptor-modulator can be used for anti-inflammation, and therapeutic treatment of autoimmune diseases and the like. Furthermore, the publication discloses test data showing that some of the compounds are CB2-selective modulators (see patent reference 34).

WO 01/32629 describes that CB2 antagonists can be used for anti-inflammation, and therapeutic treatment of immune diseases and the like (see patent reference 35).

WO01/58869 describes that CB agonists, particularly CB2 agonists can be used for therapeutic treatment of respiratory diseases, particularly asthma, bronchitis, etc. Furthermore, the publication describes that the agonists suppress mucin production of lung epithelial cells (see patent reference 36).

WO 01/96330 discloses CB2-binding compounds, exemplified indications include respiratory diseases, for example, asthma and bronchitis, and inflammatory diseases (see patent reference 37).

WO 02/10135 describes that CB2 agonists are effective for therapeutic treatment of asthma, nasal allergy, atopic dermatitis, autoimmune disease and the like. Furthermore, the publication shows test results demonstrating that the compounds suppress cAMP production (see patent reference 38).

WO 02/42269 describes that CB2 agonists are effective for therapeutic treatment of immune diseases, such as psoriasis; allergic disease, such as hypersensitivity, asthma, allergic rhinitis and contact dermatitis; inflammatory diseases, such as arthritis; etc. (see patent reference 39).

WO 02/58636 describes that cannabinoid-like compounds, particularly CB2-selective compounds can be used for anti-inflammation, regulation of immune system and so on. Furthermore, the publication describes that the compounds are agonists suppressing cAMP production (see patent reference 40).

WO 02/60447 describes CB1-selective modulators and CB2-selective modulators. Furthermore, the publication describes that the CB2-selective modulators, particularly antagonists can be used for anti-inflammation, regulation of immune system and so on (see patent reference 41).

WO 02/53543 describes that compounds with CB2 affinity can be used as anti-inflammatory agents, immunosuppressors and the like. The publication also describes that some of the compounds exert agonist actions based on a measurement of the amount of cAMP produced due to forskolin stimulation. Furthermore, it describes a test method using sheep erythrocyte-induced delayed type hypersensitivity model (see patent reference 42).

WO 02/72562 describes that compounds with CB2 affinity, particularly agonists can be used as anti-inflammatory agents, immunosuppressors and the like. The publication also describes that some of the compounds exert agonist actions based on a measurement of the amount of cAMP produced due to forskolin stimulation. Furthermore, it describes a test method using sheep erythrocyte-induced delayed type hypersensitivity model (see patent reference 43).

WO 02/62750 describes that cannabinoid modulators, particularly compounds binding to CB2 are effective for therapeutic treatment of atopic dermatitis, allergy, asthma, chronic occlusive lung diseases, bronchitis, etc. (see patent reference 44).

WO02/85866 describes that CB2-selective agonists are effective for pain treatment (see patent reference 45).

However, hitherto, none of these compounds have been verified to specifically act on CB2, and whether these compounds are really effective for therapeutic treatment of allergic diseases, particularly non-immediate type allergic diseases have not been confirmed based on reliable experiment or with theoretical evidence. Still less are there any data verifying that these compounds are effective for allergic dermatitis, atopic dermatitis, allergic asthma, immediate type asthmatic response, late asthmatic response and airway hypersensitivity. Furthermore, none of these compounds are literary taught or suggested to exhibit therapeutic effect via the action as a CB2 inverse agonist.

As described above, there are so divers findings about the relation between the action on cannabinoid receptors and pathogenesis. Particularly, no common understanding has been reached whether agonists, antagonists or inverse agonists should be used as CB2-selective modulator for clinical application.

In such circumstances, cannabinoid modulators, particularly inverse agonists, that can be used as anti-allergy agents have not yet been developed.

For the evaluation of the pharmacological actions in accordance with the present invention, the present inventors used, as pathological model animals effective for the judgment of anti-allergy effect, DNFB-induced allergic dermatitis mice with induced inflammation similar to atopic dermatitis (see non-patent reference 4), IgE-dependent allergic dermatitis model mouse with induced dermatitis (early phase, late phase of very late phase) (see non-patent reference 5) and soon. These pathological models are used as models appropriate for the evaluation of anti-allergic actions, particularly for the evaluation of pharmacological actions on atopic dermatitis.

[Patent Reference 1]

JP-A 2000-256323 (Examples 3 to 5, page 29; and page 6, right column, line 42 to page 7, left column, line 1; page 65, right column, lines 43 to 46; page 63, left column, line 16 to page 65, left column, line 37)

[Patent Reference 2]

JP-A Sho 52-113976 (page 3, lower right column, lines 1 to 4; page 8, upper right column, lines 12 to 17)

[Patent Reference 3]

JP-T 2002-511411 (page 6, column No. 0005; page 7, column No. 0009)

[Patent Reference 4]

WO 01/64212 (page 4, lines 1 to 29)

[Patent Reference 5]

WO 01/95899 (page 20, line 7 to page 23, line 23)

[Patent Reference 6]

WO 01/89589 (page 2, line 15 to page 4, line 2; FIGS. 2B and 2C)

[Patent Reference 7]

WO 00/16756 (page 13, line 18 to page 15, line 14; table, page 30, line 13 to page 32; page 43, line 4 to page 44, line)

[Patent Reference 8]

JP-T Hei 8-504195 (page 12, table II; page 16)

[Patent Reference 9]

JP-A Hei 6-73014 (page 6, left column, lines 28 to 50)

[Patent Reference 10]

JP-A Hei 7-324076 (page 8, left column, lines 4 to 34)

[Patent Reference 11]

WO 01/98289 (page 5, line 13 from the bottom to page 7, line 11; page 12, line 7 to line 13)

[Patent Reference 12]

WO 02/26702

[Patent Reference 13]

WO 01/87297 (page 3, lines 9 to 15; page 10, lines 7 to 13)

[Patent Reference 14]

WO 02/42248 (page 6, line 5 from the bottom to page 7, line 20, page 12, lines 14 to 17)

[Patent Reference 15]

WO 02/47691 (page 2, column No. 0006; page 3, line 4 to the last line)

[Patent Reference 16]

JP-T Hei 11-500411 (page 9, line 12 to page 11, line 12; page 65, line 22 to page 67, line 6)

[Patent Reference 17]

JP-T Hei 11-501615 (page 16, lines 16 to 21; page 52, line 14 to page 54, line 7)

[Patent Reference 18]

JP-T Hei 10-508870 (page 13, lines 11 to 12; page 34, lines 7 to 22)

[Patent Reference 19]

JP-T Hei 11-507937 (page 13, lines 10 to 22; page 66, line 14 to page 67, line 5)

[Patent Reference 20]

JP-T 2000-502080 (page 42, line 19 to page 44, line 2)

[Patent Reference 21]

JP-T 2001-508799 (page 14, lines 5 to 14; page 27, lines 9 to 18)

[Patent Reference 22]

JP-T 2001-516361 (page 6, line 18 to page 7, line 2; page 14, lines 17 to 18)

[Patent Reference 23]

JP-T 2001-515470 (page 86, line 7 to page 87, line 14)

[Patent Reference 24]

WO 99/57107 (page 1, line 1 to page 2, line 13; table, page 22)

[Patent Reference 25]

JP-T 2002-523395 (page 65, line 9 to page 66, line 20)

[Patent Reference 26]

JP-T 2002-523396 (page 78, line 3 from the bottom to page 80, line 8)

[Patent Reference 27]

JP-T 2002-539246 (page 53, line 5 to page 54, line 23; page 64, line 9 from the bottom to page 65, line 5)

[Patent Reference 28]

WO 01/4083 (page 50, line 9 to page 56, line 12)

[Patent Reference 29]

WO 01/19807 (page 27, line 11 to page 28, line 8; page 134, line 7 from the bottom to page 138, the last line)

[Patent Reference 30]

WO 01/29007 (page 4, lines 6 to 25; table 1, page 8)

[Patent Reference 31]

WO 01/28497 (page 1, line 4 from the bottom to page 3, line 6; page 9, lines 21 to 26)

[Patent Reference 32]

WO 01/32169 (page 3, line 18 to page 4, the last line)

[Patent Reference 33]

WO 01/28329 (page 2, line 1 to page 3, line 14)

[Patent Reference 34]

WO 01/28557 (page 2, line 5 to page 5, line 15; table, page 7)

[Patent Reference 35]

WO 01/32629

[Patent Reference 36]

WO 01/58869 (page 2, lines 1 to 8; page 44, line 4 from the bottom to page 46, line 15)

[Patent Reference 37]

WO 01/96330 (page 7, line 27 to page 8, line 9; page 56, lines 9 to 29)

[Patent Reference 38]

WO 02/10135 (page 71, line 10 to page 72, line 11)

[Patent Reference 39]

WO 02/42269

[Patent Reference 40]

WO 02/58636 (page 7, line 5 to page 8, line 25; page 29, lines 18 to 25)

[Patent Reference 41]

WO 02/60447 (page 6, line 1 to page 7, line 2; page 8, lines 7 to 17; table 1, page 9)

[Patent Reference 42]

WO 02/53543 (page 85, line 4 to the last line; page 278, line 4 to page 281, line 15)

[Patent Reference 43]

WO 02/72562 (page 29, line 22 to page 30, line 18; page 120, line 5 to page 123, line 19)

[Patent Reference 44]

WO 02/62750 (page 3, line 14 to page 4, the last line)

[Patent Reference 45]

WO 02/85866 (page 1, lines 4 to 8; page 8, line 31 to page 9, line 3)

[Non-Patent Reference 1]

Biology and Chemistry, Yamamoto Shozo, et al., ed., Vol. 39, No. 5, pp. 293-300, 2001

[Non-Patent Reference 2]

Expert Opinion on Therapeutic Patents, Vol. 12, No. 10, pp. 1475-1489, 2002

[Non-Patent Reference 3]

The Journal of Pharmacology and Experimental Therapeutics, Vol. 296, No. 2, pp. 420-425, 2001 (table 1 on page 422; table 3 on page 423)

[Non-Patent Reference 4]

Journal of Allergy Clinical Immunology, Vol. 100, No. 6, Part 2, pp. 39-44, December 1997

[Non-Patent Reference 5]

Pharmacology, Vol. 60, No. 2, pp. 97-104, February 2000

[Non-Patent Reference 6]

Munro et al., Nature, Vol. 365, pp. 61-65, 1993

### SUMMARY OF THE INVENTION

As described above, cannabinoid receptor-modulator have not yet been successfully applied as pharmacological products and effective uses thereof are being examined.

Thus, an objective of the invention is to provide a novel therapeutic agent for non-immediate type allergic diseases that contains, as the active ingredient, a cannabinoid receptor-modulator, particularly a modulator selective for peripheral cell type cannabinoid receptor (CB2), more particularly an inverse agonist.

The present inventors intensively studied to achieve the above objective, and accomplished the present invention by experimentally confirming and theorizing for the first time that a selective CB2-modulator, particularly an inverse agonist, very effectively functions against allergic diseases such as allergic asthma, atopic dermatitis, allergic rhinitis and allergic conjunctivitis. The pharmacological product of the invention is particularly effective as a therapeutic agent for allergic asthma and atopic dermatitis. This result, i.e., the effect of the present invention was unpredictable from the description of JP-A2000-256323 (WO 00/40562) described above, and even surprising for the present inventors.

Specifically, the present invention provides:

[1] a therapeutic agent for a non-immediate-type allergic disease, which comprises as an active ingredient an inverse agonist of the peripheral cell type cannabinoid receptor;

[2] the therapeutic agent for a non-immediate-type allergic disease according to [1], wherein the inverse agonist is a compound that exhibits the inverse agonistic action by antagonizing the agonistic action of 2-arachidonoylglycerol (2-AG) and/or 2-arachidonoylglycerol ether (2-AG-E);

[3] the therapeutic agent for a non-immediate-type allergic disease according to [1], wherein the inverse agonist is a compound selected from the group consisting of: compound A, compound B, compound C, compound D, compound E, compound F, compound G, compound H, compound I and SR144528 shown below, and pharmaceutically acceptable salts thereof:

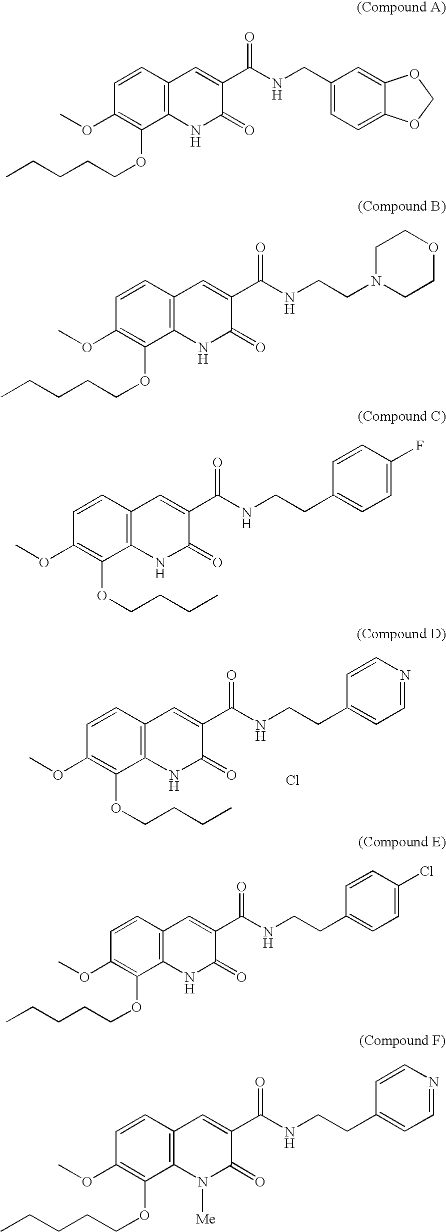

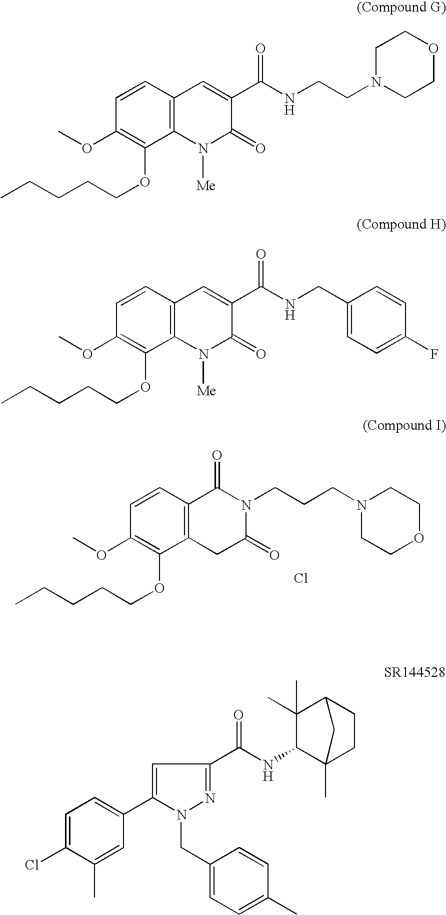

[4] the therapeutic agent for a non-immediate-type allergic disease according to [1], wherein the non-immediate-type allergic disease is allergic dermatitis, allergic asthma, allergic rhinitis and/or allergic conjunctivitis;

[5] the therapeutic agent for a non-immediate-type allergic disease according to [4], wherein the non-immediate-type allergic disease is allergic dermatitis;

[6] the therapeutic agent for a non-immediate-type allergic disease according to [4], wherein the non-immediate-type allergic disease is allergic asthma;

[7] the therapeutic agent for a non-immediate-type allergic disease according to [6], wherein the allergic asthma is a late asthmatic response and/or airway hypersensitivity;

[8] the therapeutic agent for a non-immediate-type allergic disease according to [1], wherein the non-immediate-type allergic disease is a disease with late phase allergic reaction and/or delayed-type allergic reaction;

[9] the therapeutic agent for a non-immediate-type allergic disease according to [1], wherein the inverse agonist of the peripheral cell type cannabinoid receptor is a compound that also has a leukotriene-inhibiting effect;

[10] the therapeutic agent for a non-immediate-type allergic disease according to [1], wherein the non-immediate-type allergic disease is a disease associated with 2-AG and/or 2-AG-E;

[11] a method for identifying a candidate compound for a therapeutic agent for a non-immediate-type allergic disease, which comprises the steps of:

(a) contacting a test compound with a cannabinoid receptor and an endogenous cannabinoid;

(b) determining the binding activity of the cannabinoid receptor to the endogenous cannabinoid; and

(c) selecting the compound that decreases the binding activity determined in step (b) compared with the activity determined in the absence of the test compound;

[12] the method according to [11], wherein the cannabinoid receptor is CB2 and the endogenous cannabinoid is 2-AG or 2-AG-E;

[13] a method for identifying a candidate compound for a therapeutic agent for a non-immediate-type allergic disease, which comprises the steps of:

(a) selecting candidate compounds that selectively bind to CB2;

(b) selecting a compound that is a CB2 inverse agonist among the compounds selected in step (a); and

(c) determining the anti-allergic activity of the compound selected in step (b);

[14] a method for treating a non-immediate-type allergic disease, which comprises administering a preparation containing an effective amount of CB2 inverse agonist to a patient affected with the non-immediate-type allergic disease;

[15] the method for treating a non-immediate-type allergic disease according to [14], wherein the inverse agonist is a compound that exhibits the inverse agonistic action by antagonizing the agonistic action of 2-AG and/or 2-AG-E;

[16] the method for treating a non-immediate-type allergic disease according to [14], wherein the inverse agonist is a compound selected from the group consisting of: compound A, compound B, compound C, compound D, compound E, compound F, compound G, compound H, compound I and SR144528, and pharmaceutically acceptable salts thereof:

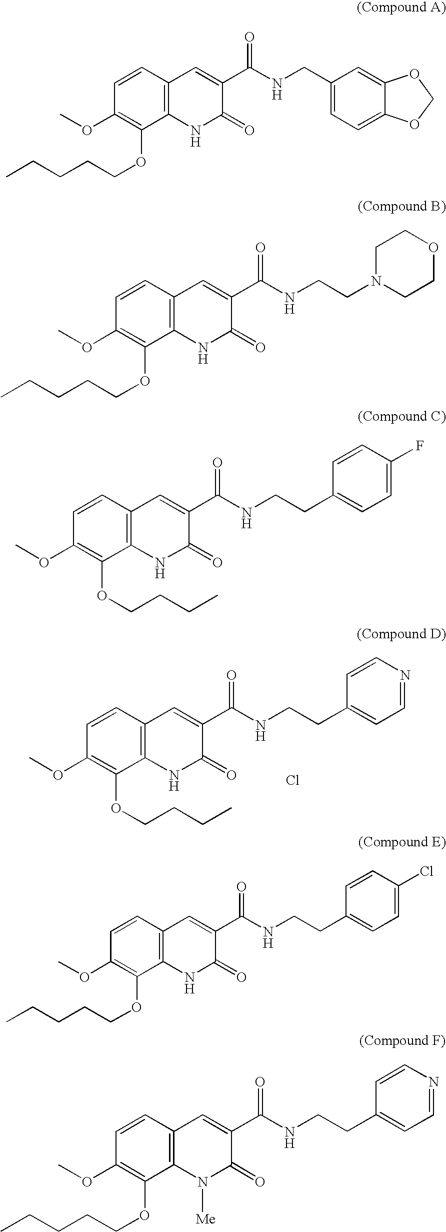

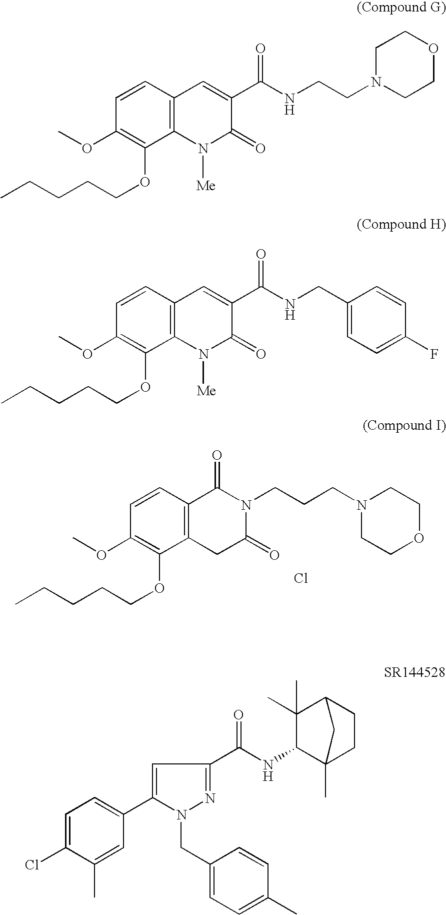

[17] the method for treating a non-immediate-type allergic disease according to [14], wherein the non-immediate-type allergic disease is selected from the group consistingof: allergic dermatitis, allergic asthma, allergic rhinitis and/or allergic conjunctivitis;

[18] a therapeutic agent for a disease associated with 2-AG and/or 2-AG-E, which comprises as an active ingredient an inverse agonist of the peripheral cell type cannabinoid receptor; and

[19] the therapeutic agent according to [18], wherein the disease associated with 2-AG and/or 2-AG-E is selected from the group consisting of: hematologic malignancies, sepsis and diseases of circulatory system.

### BRIEF DESCRIPTION OF THE DRAWINGS

FIG. 1 shows a graph depicting the influence of the test compound on ear swelling after the fourth antigen application in murine DNFB-induced allergic dermatitis model. The ordinate shows “the increase of the thickness of the ear (×10⁻² mm)” of the mouse after the dosing of the test compound. The results of sham treatment, vehicle (oral dosing of 10 mL/kg vehicle alone), prednisolone (oral dosing of 1, 2 and 5 mg/kg as positive control) and compound A (oral dosing of 0.1, 1 and 10 mg/kg) are shown from the left.

FIG. 2 shows a graph depicting the influence of the test compound on ear swelling after a fifth antigen application on murine DNFB-induced allergic dermatitis. The ordinate shows “the increase of the thickness of the ear (×10⁻² mm)” of the mouse after the dosing of the test compound. The results of sham treatment, vehicle (oral dosing of 10 mL/kg vehicle alone), prednisolone (oral dosing at 1, 2 and 5 mg/kg as positive control) and compound A (oral dosing at 0.1, 1 and 10 mg/kg) are shown from the left.

FIG. 3 shows a graph depicting the influence of the test compound on the spleen wet weight in the DNFB-induced allergic dermatitis models. The ordinate shows the wet weight of the spleen (mg). The results of sham treatment, vehicle (oral dosing of 10 mL/kg vehicle alone), prednisolone (oral dosing at 1, 2 and 5 mg/kg as positive control) and compound A (oral dosing at 0.1, 1 and 10 mg/kg) are shown from the left.

FIG. 4 shows a graph depicting the influence of the test compound on ear swelling in the early phase (one hour after antigen application) of murine IgE-dependent allergic dermatitis reaction. The ordinate shows “the increase of the thickness of the ear (×10⁻² mm)” of the mouse after the dosing of the test compound. The results of vehicle (oral dosing of 10 mL/kg vehicle alone), ketotifen fumarate (oral dosing at 10 mg/kg as positive control), pranlukast hydrate (oral dosing at 30 mg/kg as positive control) and compound A (oral dosing at 10 mg/kg) are shown from the left.

FIG. 5 shows a graph depicting the influence of the test compound on ear swelling in the late phase (24 hours after antigen application) of murine IgE-dependent allergic dermatitis reaction. The ordinate shows “the increase of the thickness of the ear (×10⁻² mm)” of the mouse after the dosing of the test compound. The results of vehicle (oral dosing of 10 mL/kg vehicle alone), ketotifen fumarate (oral dosing at 10 mg/kg as positive control), pranlukast hydrate (oral dosing at 30 mg/kg as positive control) and compound A (oral dosing at 10 mg/kg) are shown from the left.

FIG. 6 shows a graph depicting the influence of the test compound on ear swelling in the very late phase (8 days after antigen application) of murine IgE-dependent allergic dermatitis reaction. The ordinate shows “the increase of the thickness of the ear (×10⁻² mm)” of the mouse after the dosing of the test compound. The results of vehicle (oral dosing of 10 mL/kg vehicle alone), ketotifen fumarate (oral dosing at 10 mg/kg as positive control), pranlukast hydrate (oral dosing at 30 mg/kg as positive control) and compound A (oral dosing at 10 mg/kg) are shown from the left.

FIG. 7 shows a graph depicting the influence of the dosing period of the test compound in murine IgE-dependent allergic dermatitis reaction. The ordinate shows “the increase of the thickness of the ear (×10⁻² mm)” of the mouse after the dosing of the test compound. The symbol 0-8 represents a dosing period from the antigen application day to the 8th day. The results of vehicle (oral dosing of 10 mL/kg vehicle alone for 9 days) and compound A (oral dosing at 10 mg/kg for 9, 8, 7, 5 and 3 days) are shown from the left.

FIG. 8 shows a graph depicting the influence of the test compound on the respiratory resistance during immediate type asthma (one minute after antigen challenge) of antigen-induced asthma in guinea pigs. The ordinate shows % increase of airway resistance (sRaw). The results of sham treatment, vehicle (oral dosing of 10 mL/kg solvent alone), compound A (oral dosing at 10, 30 and 100 mg/kg), pranlukast (oral dosing at 30 mg/kg as positive control) and prednisolone (oral dosing at 30 mg/kg as positive control) are shown from the left.

FIG. 9 shows a graph depicting the influence of the test compound on the respiratory resistance during late asthmatic response (4 to 8 hours after antigen challenge) of antigen-induced asthma in guinea pigs. The ordinate shows AUC₄₋₈ₕᵣ (% hr). AUC₄₋₈ₕᵣ denotes the increase (area under the curve) of the airway resistance (sRaw) over 4 to 8 hours after the antigen challenge. The results of sham treatment, vehicle (oral dosing of 10 mL/kg solvent alone), compound A (oral dosing at 10, 30 and 100 mg/kg), pranlukast (oral dosing at 30 mg/kg as positive control) and prednisolone (oral dosing at 30 mg/kg as positive control) are shown from the left.

FIG. 10 shows a graph depicting the influence of the test compound on airway hypersensitivity in guinea pigs. The ordinate shows PC₁₀₀ ACh (mg/mL). PC₁₀₀ ACh denotes the concentration of acetylcholine required for airway resistance (sRaw) after acetylcholine inhalation to achieve 100% increase from the sRaw after physiological saline inhalation. The results of sham treatment, vehicle (oral dosing of 10 mL/kg solvent alone), compound A (oral dosing at 10, 30 and 100 mg/kg), pranlukast (oral dosing at 30 mg/kg as positive control) and prednisolone (oral dosing at 30 mg/kg as positive control) are shown from the left.

FIG. 11 shows a graph depicting the influence of the test compound on leukotriene production from human basophils. The ordinate shows the amount (pg/mL) of leukotrienes (C4/D4/E4) and the abscissa the amount (mg/mL) of anti-IgE antibody.

FIG. 12 shows a graph depicting the influence of the test compound on the production of leukotrienes from rat mast cells. The ordinate expresses the amount (pg/mL) of leukotrienes (C4/D4/E4) and the abscissa the amount (ng/mL) of DNP-BSA.

FIG. 13 shows graphs depicting the influence of the test compound on ear swelling in the very late phase (8 days after antigen application) of murine IgE-dependent allergic dermatitis reaction. The ordinate shows “the thickening of the ear (×10⁻² mm)” of the mouse after the dosing of the test compound. In the upper panel, the results of the non-sensitized group, the sensitized group, HU-308 (oral dosing at 10 and 50 mg/kg), compound A (oral dosing at 0.1, 1 and 10 mg/kg) and prednisolone (oral dosing at 5 mg/kg as positive control) are shown from the left. In the lower panel, the results of the non-sensitized group, the sensitized group, SR144528 (oral dosing at 0.1, 1 and 10 mg/kg), compound A (oral dosing at 10 mg/kg) and prednisolone (oral dosing at 5 mg/kg as positive control) are shown from the left. Mean ± standard error (n=8). **: p<0.01, ***: P<0.001 (vs sensitized group; Dunnett test), ###: p<0.001 (vs sensitized group; Student's t-test) and $$$: p<0.001 (vs non-sensitized group; Student's t-test).

FIG. 14 shows graphs depicting the influence of the test compound on the wet weights of the spleen and thymus in the very late phase (8 days after antigen application) of the murine IgE-dependent allergic dermatitis models. The ordinate shows the wet weight (mg) of the spleen in the upper panel. The ordinate in the lower panel shows the wet weight (mg) of the thymus. Both panels show from the left the results of the non-sensitized group, the sensitized group, HU-308 (oral dosing at 10 and 50 mg/kg), compound A (oral dosing at 0.1, 1 and 10 mg/kg) and prednisolone (oral dosing at 5 mg/kg). Mean ± standard error (n =8). *: p<0.05, ***: ##: p<0.01, ###: p<0.001 (vs sensitized group; Student's t-test) and $$: p<0.01 (vs non-sensitized group; Student's t-test).

FIG. 15 shows graphs depicting the influence of the test compound on the wet weights of the spleen and thymus in the very late phase (8 days after antigen application) of the murine IgE-dependent allergic dermatitis models. The ordinate in the upper panel shows the wet weight (mg) of the spleen. The ordinate in the lower panel shows the wet weight (mg) of the thymus. Both of the panels show from the left the results of the non-sensitized group, the sensitized group, SR144528 (oral dosing at 0.1, 1 and 10 mg/kg), compound A (oral dosing at 10 mg/kg) and prednisolone (oral dosing at 5 mg/kg). Mean ± standard error (n=8). ###: P<0.001 (vs sensitized group; Student's t-test).

FIG. 16 shows a graph depicting the overtime change of ear swelling induced by 2-AG and so on. The ordinate shows the thickening of the ear (×10⁻² mm) of the mouse after the dosing of the test compound. The abscissa shows the course of the time after the application of the compound from the left. Mean ± standard error (n=6).

FIG. 17 shows a graph depicting the effect of compound A on ear edema induced by 2-arachidonoylglycerol ether (2-AG-E). The ordinate shows the AUC (on day 0 to day 8). The graph shows from left the results of sham treatment, vehicle (oral dosing of 10 mg/kg solvent alone) and compound A (oral dosing at 0.01, 0.1, 1 and 10 mg/kg). Mean ±standard error (n=8). **: p<0.01, ***: P<0.001 (vs solvent group; Dunnett test) and $$$: p<0.001 (vs false treatment group; Student's t-test).

FIG. 18 shows a graph depicting the effect of the test compound on spontaneous scratching reaction of NC mice. The ordinate shows the number of scratching movements (movements/hour) of the mice before and after the dosing of the test compound. The graph shows from the left the results of vehicle (oral dosing of solvent alone), compound A (oral dosing at 1 and 10 mg/kg), tacrolimus hydrate (oral dosing at 1 mg/kg) and betamethasone valerate (oral dosing at 1 mg/kg).

FIG. 19 shows a graph depicting the IgE-dependent triphasic ear swelling in wild-type and CB2 knockout mice. The ordinate shows the increase of the thickness of the ear (10⁻² mm). The abscissa shows the time course after the test. Each data was determined using for to eight mice and given as mean ± standard error. *: p<0.05, **: p<0.01, ***: p<0.001 (vs sensitized wild-type; Student's t-test), ##: p<0.01 and ###: p<0.001, (vs non-sensitized CB2-knockout; Student's t-test)

FIG. 20 shows a graph depicting the 2-AG-E-induced ear swelling in wild-type and CB2 knockout mice. The ordinate shows the increase of the thickness of the ear (10⁻² mm). The abscissa shows the time course after 2-AG-E treatment. The ear swelling was induced by topical application of 10 μL/site of 2-AG-E (100 mg/mL) on both sides of the right ear. Each data was determined using eight mice and given as mean ± standard error. **: p<0.01 and NS: no significant difference (vs wild-type mice, Student's t-test).

FIG. 21 shows graphs depicting 2-AG-E-induced ear swelling in wild-type and CB2 knockout mice. The upper panel shows the result obtained 0 to 24 hours after the treatment; and the lower panel shows the result obtained 1 to 8 days after the treatment. The ordinate indicates the AUC (time ×10⁻² mm). Both of the panels show form the left the results of wild-type and CB2 knockout mice. **: p<0.01 and NS: no significant difference (vs wild-type mice, Student's t-test).

FIG. 22 shows a graph depicting the 2-AG-E-induced ear swelling in wild-type (WBB6F1-+/+) and mast cell-deficient mice (WBB6F1-W/Wv) The ordinate shows the increase in the thickness of the ear (10⁻² mm). The abscissa shows the time course after the 2-AG-E treatment. The ear swelling was induced by topical application of 10 μL/site of 2-AG-E (100 mg/mL) on both sides of the right ear. Each data was determined using eight mice and given as mean ± standard error. *: p<0.05, **: p<0.01 and ***: p<0.001 (vs WBB6F1-+/+ mice; Student's t-test).

FIG. 23 shows graphs depicting the 2-AG-E-induced ear swelling in normal mice and mast cell-deficient mice (WBB6F1-W/Wv). The upper panel shows the result obtained 0 to 24 hours after the treatment; and the lower panel shows the result obtained 1 to 8 days after the treatment. The ordinate shows the AUC (time ×10⁻² mm). Both of the panels show, from the left, the results of WBB6F1-+/+ (acetone, 2-AG-E) and WBB6F1-W/W^{v} (acetone, 2-AG-E).

### DETAILED DESCRIPTION OF THE INVENTION

The terms used in the present specification are described below.

The term “cannabinoid receptor modulator” refer to a substance that regulate the biological activity of cannabinoid receptors or a substance that regulates the expression of cannabinoid receptors.

The former includes agonists, antagonists, inverse agonists and other substances that enhance or reduce the sensitivity of cannabinoid receptors. The latter includes substances that enhance or suppress the expression of genes of cannabinoid receptors.

The term “agonist” refers to a substance that induces receptor-mediated intracellular signaling, and “antagonist” refers to a substance that antagonistically reduces the action of the agonist.

The term “inverse agonist” refers to a substance that exhibits an opposite action to the original activity of a receptor agonist. Inverse agonists include those acting by antagonizing an endogenous ligand or drug, and those without being influenced by endogenous ligands or drugs. For example, in relation to the cannabinoid receptor, cannabinoid has been reported to suppress the increased cAMP level, whereas compound A was found to elevate the cAMP level. More specifically, endogenous cannabinoids, 2-AG and 2-AG-E (non-selective CB agonists), suppress the forskolin-stimulated cAMP production in hCB2-expressing CHO cells. A substance that functions to increase cAMP production can be regarded as an inverse agonist. Such inverse agonists specifically include compound A, compound B, compound C, compound D, compound E, compound F, compound G, compound H, compound I, SR144528 and AM630. Preferred inverse agonists are compound A and SR144528.

Cannabinoid receptor-modulator specifically include: compounds represented by general formula [I] of JP-A 2000-256323 (WO 00/40562), more specifically 2-oxoquinoline compounds such as N-(benzo[1,3]dioxol-5-ylmethyl)-7-methoxy-2-oxo-8-pentyloxy-1,2-dihydroquinoline-3-carboxamide (compound A); compounds described in WO 01/28557, such as Δ9-THC, Nabilone (LY-109514), CP-55940, PRS-211096, PRS-211335, PRS-211359, SR144528, SR141716, Rimonabant (SR141716A), SR14778, AMG-3, SLV-319, AM-251, AM-281, AM374, AM404, AM630, AM-694, AM2233, AM2230 and AM1221; compounds described in WO 01/28497, such as AM1703; compounds described in WO 01/28329, such as AM1710; compounds described in WO 01/32169 such as HU-308; compounds described in WO 99/51560 such as HU-310; compounds described in WO 96/02248, such as JWH-051, JWH-161, O-1236, O-1057, O-2093, L-759633, L-759656, L-768242 and LY320135; BAY-38-7271; compounds described in WO 02/24630; compounds described in WO 02/10135; compounds described in WO 01/96330; compounds described in WO 01/85092; compounds described in WO 01/74763; compounds described in WO 01/70700; compounds described in WO 01/64634; compounds described in WO 01/64633; compounds described in WO 01/64632; compounds described in WO 01/58869; compounds described in WO 01/58445; compounds described in WO 01/04083; compounds described in WO 01/32629; compounds described in WO 01/29007; compounds described in WO 01/28588; compounds described in JP-T 2001-515470 (U.S. Pat. No. 6,262,112); compounds described in JP-T 2002-539246 (WO 00/56303); compounds described in WO 00/46209; compounds described in WO 00/32200; compounds described in WO 00/16756; compounds described in WO 00/15609; compounds described in JP-T 2002-523396 (WO 00/10968); compounds described in JP-T 2002-523395 (WO 00/10967); compounds described in WO 99/60987; compounds described in WO 99/57107; compounds described in WO 99/57106; compounds described in WO 99/52524; compounds described in WO 99/26612; compounds described in WO 99/24471; compounds described in WO 99/2499; compounds described in JP-T 2001-516361 (WO 98/41519); compounds described in WO 98/37061; compounds described in WO 98/32441; compounds described in JP-T 2001-508799 (WO 98/31227); compounds described in WO 97/29079; compounds described in JP-T 2000-502080 (WO 97/21682); compounds described in WO 97/19063; compounds described in JP-T Hei 11-507937 (WO 97/860); compounds described in WO 96/20268; compounds described in JP-T -Hei 10-508870 (WO 96/25397); compounds described in JP-T Hei 11-501615 (WO 96/18600); compounds described in JP-T Hei 11-500411 (WO 96/18391); compounds described in WO 94/12466; compounds described in U.S. Pat. No. 6,284,788; compounds described in U.S. Pat. No. 5,939,429; compounds described in U.S. Pat. No. 5,804,592; compounds described in U.S. Pat. No. 5,605,906; compounds described in U.S. Pat. No. 5,624,941; compounds described in U.S. Pat. No. 5,462,960; compounds described in U.S. Pat. No. 5,081,122; compounds described in U.S. Pat. No. 5,013,837; compounds described in DE 10015866; compounds described in DE 19837627; compounds described in DE 19837638; compounds described in WO 01/58450; compounds described in WO 01/32663; compounds described in WO 01/28498; compounds described in WO 01/24798; compounds described in FR 2805818; compounds described in FR 2805817; compounds described in FR 2805810; compounds described in FR 2799124; compounds described in FR 2789079; compounds described in FR 2789078; compounds described in WO 01/89589; compounds described in WO 01/95889; compounds described in WO 01/98289; compounds described in WO 02/19383; compounds described in WO 02/26702; compounds described in WO 02/28346; compounds described in WO 01/87297; compounds described in WO 02/36590; compounds described in WO 02/42269; compounds described in WO 02/42248; compounds described in WO 02/47691; compounds described in WO 02/58636; compounds described in WO 02/60447; compounds described in WO 02/65997; compounds described in WO 02/53543; compounds described in WO 02/72562; compounds described in WO 02/62750; compounds described in WO 02/80903; and compounds described in WO 02/85866.

Preferably, the cannabinoid receptor-modulator selectively act on peripheral cell type cannabinoid receptors including: the compounds described in JP-A 2000-256323 (WO 00/40562); the compounds described in WO 02/10135; the compounds described in WO 01/28557, such as SR 144528, AM 630 and AM1221; the compounds described in WO 01/28497, such as AM1703; the compounds described in WO 01/28329, such as AM1710; the compounds described in WO 01/32169, such as HU-308; JWH-051; L-759633; L-759656; L-768242; the compounds described in WO 01/74763; the compounds described in WO 01/32629; the compounds described in WO 01/29007; the compounds described in WO 01/19807; the compounds described in WO 01/4083; the compounds described in JP-T 2002-539246 (WO 00/56303); the compounds described in JP-T 2002-523396 (WO 00/10968); the compounds described in JP-T 2002-523395 (WO 00/10967); the compounds described in WO 99/57107; the compounds described in WO 99/2499; the compounds described in JP-T 2001-516361 (WO 98/41519); the compounds described in JP-T 2001-515470 (U.S. Pat. No. 6,262,112); the compounds described in JP-T 2001-508799 (WO 98/31227); the compounds described in WO 97/29079; the compounds described in JP-T 2000-502080 (WO97/21682); the compounds described in JP-T Hei 11-507937 (WO 97/860); the compounds described in JP-T Hei 10-508870 (WO 96/25397); the compounds described in JP-T Hei 11-501615 (WO 96/18600); the compounds described in JP-T Hei 11-500411 (WO 96/18391); the compounds described in U.S. Pat. No. 5,605,906; the compounds described in WO 01/58869; the compounds described in WO 01/96330; the compounds described in WO 02/10135; the compounds described in WO 02/42269; the compounds described in WO 02/58636; the compounds described in WO 02/60447; the compounds described in WO 02/53543; the compounds described in WO 02/72562; the compounds described in WO 02/62750; and the compounds described in WO 02/85866. More preferable compounds include: the compounds described in JP-A 2000-256323 (WO 00/40562); the compounds described in WO 01/28557, such as SR144528, AM 630 and AM1221; the compounds described in WO 01/28497 such as AM1703; the compounds described in WO 01/28329, such as AM1710; the compounds described in WO01/32169, such as HU-308; JWH-051; L-759633; L-759656; L-768242; the compounds described in WO 01/32629; the compounds described in WO1/29007; and the compounds described in WO98/41519. Particularly preferable compounds include the 2-oxoquinoline compound, N-(benzo[1,3]dioxol-5-ylmethyl)-7-methoxy-2-oxo-8-pentyloxy-1,2-dihydroquinoline-3-carboxamide (compound A). More preferable are compound A, compound B, compound C, compound D, compound E, compound F, compound G, compound H, compound I, SR144528 and AM630. Still more preferable are compound A and SR144528, and most preferable is compound A.

Herein, “allergic diseases” include, but are not limited to, anaphylaxis, digestive tract allergy, allergic gastritis, allergic dermatitis, dermatitis such as rash against Japanese lacquer (urushi) and rash against cosmetics, urticaria, atopic dermatitis, asthma, allergic asthma, atopic asthma, allergic bronchial pulmonary aspergillosis, pollenosis, allergic rhinitis, allergic conjunctivitis, allergic sarcoma angitis, chemical allergy, serum disease, post-organ transplantation rejection, tuberculosis lesion and post-organ transplantation rejection. The term “allergic disease” may be used for any disease as long as it relates to allergy. More preferably, allergic diseases of the present invention include allergic dermatitis, atopic dermatitis, asthma, allergic asthma, atopic asthma, allergic rhinitis and allergic conjunctivitis. Particularly preferably, the allergic diseases include allergic disease of skin or respiratory organs. More specific indication of the present invention includes allergic dermatitis, atopic dermatitis, allergic asthma and atopic asthma.

The term “non-immediate-type allergic disease” refers to a disease involving late phase allergic reaction and/or delayed-type allergic reaction, and may be accompanied with immediate-type allergic reaction. For example, non-immediate-type allergic diseases include diseases only related to the delayed-type allergic reaction, diseases related to both the late phase and delayed type allergic reactions, and diseases related to the immediate-type, late phase and delayed-type allergic reactions.

Herein, the term “immediate-type allergic reaction” refers to an allergic reaction which is caused within 30 minutes, at least within two hours after invasion of antigens.

The term “late phase allergic reaction” refers to an allergic reaction which is caused after a few hours to a few days, more specifically, after two hours to two or three days.

The term “delayed-type allergic reaction” refers to an allergic reaction which is caused after a few days or later.

In a triphasic dermatitis model, the “early phase” and the “late phase” correspond to the time of onsets of immediate-type allergic reaction and late phase allergic reaction, respectively. The “very late phase” corresponds to the allergic reaction that occurs after 4 days or later, preferably after 6 to 8 days.

Non-immediate-type allergic diseases specifically include, in addition to allergic dermatitis, atopic dermatitis, allergic asthma and atopic asthma, hemolytic anemia, thrombocytopenic purpura, myasthenia gravis, Goodpasture syndrome, acute glomerulonephritis induced by hemolytic streptococcus, rheumatoid arthritis and connective tissue diseases, serum disease, viral hepatitis, allergic alveolitis, tuberculin reaction, tuberculosis lesion, post-organ transplantation rejections, dermatitis such as Japanese lacquer or rush against cosmetics, late-type asthma, etc., as well as anaphylaxis, allergic rhinitis, allergic conjunctivitis, pollenosis, urticaria, allergic gastroenteritis and itch that involve late phase and/or delayed-type allergic reactions. Diseases that can be treated or prevented particularly include allergic dermatitis, atopic dermatitis, allergic asthma and atopic asthma.

The term “allergic dermatitis” refers to dermatitis related with allergic reaction and includes, for example, atopic dermatitis. Allergic dermatitis is discriminated from non-allergic dermatitis such as dermatitis due to injuries or wounds. As a “therapeutic agent for atopic dermatitis”, those that show therapeutic effect by acting on the allergic reaction occurring in atopic dermatitis are preferable. Furthermore, the therapeutic agent of atopic dermatitis preferably has an effect on the late phase response, the delayed-type response, or the late phase and delayed-type responses of the allergic reaction. More preferably, the therapeutic agent of atopic dermatitis has an effect on the late phase response, the delayed-type response or the late phase response and the delayed-type response, in addition to the immediate-type response.

The term “allergic asthma” refers to the allergic aspect of asthma among asthma symptoms and includes, for example, mixed type asthma and atopic asthma. Allergic asthma is discriminated from non-allergic asthma such as aspirin asthma. A “therapeutic agent for asthma” preferably exerts a therapeutic effect via the action on the allergic reaction of asthma. Furthermore, the therapeutic agent of asthma preferably exerts an effect on chronic bronchitis or airway hypersensitivity. More preferably, the therapeutic agent of asthma has an effect on chronic bronchitis and airway hypersensitivity. The therapeutic agent of asthma exerts an effect on the late phase response, the delayed-type response, or the late phase and the delayed-type responses of the allergic reaction. Still more preferably, the therapeutic agent of asthma exerts an effect on the late phase response, the delayed-type response or the late phase and the delayed-type responses, in addition to the immediate-type response.

The phrase “disease associated with 2-AG and/or 2-AG-E” refers to a disease that is caused by 2-AG and/or 2-AG-E or whose symptoms are exacerbated by 2-AG and/or 2-AG-E. For example, such disease includes the non-immediate-type allergic disease described above. Also included are hematologic malignancies, sepsis, diseases of circulatory system, etc.

The term “antipruritic action” refers to an effect that reduces scratching action, and reduces psychological stress due to itching, which effect is caused by reducing itching or eliminating itching. Preferably, the antipruritic action is not achieved by a central action, and the causes of itching are eliminated by actions such as anti-histamine action or anti-substance P action. Furthermore, the therapeutic agent preferably has an antipruritic action for allergic disease, particularly atopic dermatitis.

The “cannabinoid receptor-modulator”, “cannabinoid receptor inverse agonists” and “cannabinoid receptor antagonists” of the present invention can serve as safe pharmaceutical agents without “immunosuppressive action that causes side effects” as observed in the use of steroidal agents and immunosuppressive agents. The “immunosuppressive action that causes side effects” includes hyperkalemia, leucopenia, thrombocytopenia and such due to functional disorders of kidney and spleen. The decrease of spleen weight serves as an index for such action. None of these side effects were observed for the “cannabinoid receptor inverse agonists” of the present invention.

When pharmaceutical agents can be orally administered due to the lack of notable side effects, it can be readily handled compared with ointments, injections and the like.

Herein, a “therapeutic treatment” for allergic diseases means suppression of allergic reactions or amelioration of allergic symptoms, and includes the prevention of potential allergic reactions or allergic diseases and the prevention of the exacerbation of the disease.

The phrase “pharmaceutically acceptable salt thereof” refers to any salts of a compound, as long as the salt formed with the compound is nontoxic. Such salts can be obtained by reacting the compound, for example, with an inorganic acid, such as hydrochloric acid, sulfuric acid, phosphoric acid and hydrobromic acid; an organic acid, such as oxalic acid, malonic acid, citric acid, fumaric acid, lactic acid, malic acid, succinic acid, tartaric acid, acetic acid, trifluoroacetic acid, gluconic acid, ascorbic acid, methylsulfonic acid and benzylsulfonic acid; an inorganic base, such as sodium hydroxide, potassium hydroxide, calcium hydroxide, magnesium hydroxide and ammonium hydroxide; an organic base, such as methylamine, diethylamine, triethylamine, triethanolamine, ethylenediamine, tris (hydroxymethyl) methylamine, guanidine, choline and cinchonine; an amino acid, such as lysine, arginine and alanine. The salts also include hydrates and solvates of the compound.

The present invention also provides a method for identifying a candidate compound for a therapeutic agent for a non-immediate type allergic disease, which comprises the steps of:

(a) contacting a test compound with a cannabinoid receptor and an endogenous cannabinoid;

(b) determining the binding activity of the cannabinoid receptor to the endogenous cannabinoid; and

(c) selecting the compound that decreases the binding activity determined in step (b) compared with the activity determined in the absence of the test compound.

The assay for the binding activity in step (b) described above can be appropriately carried out by those skilled in the art according to routine methods, for example, utilizing the binding assay or measuring the cAMP concentration. More specifically, the binding activity described above can be determined by the method described in following Examples.

There is no limitation on the type of test compounds. Exemplary test compounds include various natural and artificial compounds, proteins, and random peptide groups prepared by the phage-display method, etc. Furthermore, culture supernatants of microorganisms, and natural components derived from plants or marine organisms can also be used in the method of the present invention. In addition, tissue extracts, cell extracts, expression products of gene libraries may also be used as the test compound, but is not limited to these examples.

The cannabinoid receptor used in the method described above preferably is CB2 and a preferable endogenous cannabinoid is 2-AG or 2-AG-E, but is not limited to these examples.

The present invention also provides a method for identifying a candidate compound for a therapeutic agent for a non-immediate type allergic disease, which comprises the steps of:

(a) selecting candidate compounds that selectively bind to CB2;

(b) selecting a compound that is a CB2 inverse agonist among the compounds selected in step (a); and (c) determining the anti-allergic activity of the compound selected in step (b).

In step (c) described above, those skilled in the art can determine the anti-allergic activity of a compound by an appropriate method depending on the type of allergy.

An cannabinoid receptor inverse agonist is effective as a therapeutic agent for allergic diseases, such as asthma and atopic dermatitis. Particularly, an inverse agonist that selectively acts on peripheral cell type cannabinoid receptors (CB2) can serve as effective and safe pharmaceutical agents to treat chronic and/or intractable allergic diseases and non-immediate-type allergic diseases on which existing therapeutic agents for allergic diseases have only poor effect.

### EXAMPLES

The present invention is described in detail below with reference to Examples, but is not construed as being limited thereto.

### Preparation Example 1

### Production of Capsules

| | |
|---|---|
| 1. Compound A | 30 g |
| 2. Particle cellulose | 10 g |
| 3. Lactose | 19 g |
| 4. Magnesium stearate | 1 g |
| Total | 60 g |

The ingredients 1 through 4 are mixed together and filled in a gelatin capsule.

### Preparation Example 2

### Production of Tablets

| | |
|---|---|
| 1. Compound A | 30 g |
| 2. Lactose | 50 g |
| 3. Corn starch | 15 g |
| 4. Carboxymethyl cellulose calcium | 44 g |
| 5. Magnesium stearate | 1 g |
| Total | 140 g |

Total volumes of ingredients 1 to 3 and 30 g of ingredient 4 are kneaded with water, dried under vacuum and granulated. 14 g of ingredient 4 and ingredient 5 are mixed thereto, and then tableted with a tableting machine. 1000 tablets each containing 30 mg of compound A are obtained.

In case where the compound of the present invention is used as a pharmaceutical composition, the compound can be directly administered to patients. Alternatively, the compound may be formulated into a preparation by known pharmaceutical methods for the administration. For example, if necessary, in addition to the Preparation Example 1 (capsule) and the Preparation Example 2 (tablet), the compound can be administered orally or parenterally as microcapsules, soft and hard capsules, pills, liquids, powders, granules, fine granules, film coated preparations, pellets, troches, sublingual preparations, chewing preparations, buccal preparations, pastes, syrups, suspensions, elixirs, emulsions, eye drops, ear drops, liniments, ointments, hard ointments, cataplasm, TTS preparations, lotions, inhalants and aerosol, or parenterally as injections of sterile solutions or suspensions/emulsions in water or other pharmacologically acceptable solutions. Other forms for parenteral administration include external liquid preparations and suppositories, pessaries and emulsion effervescing agents for enteric dosing, which contain one or more active substances and are formulated by routine methods. Furthermore, the compound may be appropriately combined and mixed, for example, with pharmacologically acceptable carriers or media, specifically including sterile water, physiological salines, vegetable oils, solvents, bases, emulsifiers, suspending agents, surfactants, stabilizers, flavors, aromatic agents, excipients, vehicles, preservatives, binders, diluents, isotonic agents, soothing agents, fillers, disintegrators, buffers, coating agents, lubricants, coloring agents, sweeteners, viscous agents, corrigents, solubilizers, other additives and the like, in an unit dosage form according to generally accepted pharmaceutical practice to form preparations.

Additives to be mixed into tablets and capsules include, for example, binders such as gelatin, corn starch, tragacanth gum and gum arabic; excipients such as crystal cellulose; expansion agents such as corn starch, gelatin and alginic acid; lubricants such as magnesium stearate; sweeteners such as sucrose, lactose and saccharin; and flavors such as peppermint, Gaultheria adenothris oil and cherry. When the unit dosage form is capsule, it may contain liquid carriers such as fats and oils in addition to the aforementioned materials. A sterile composition for injections can be formulated according to general pharmaceutical practice using vehicles such as distilled water for injections.

An aqueous solution for injection includes, for example, physiological saline, isotonic solutions containing glucose and other auxiliary agents such as D-sorbitol, D-mannose, D-mannitol and sodium chloride, which may be used in combination with appropriate solubilizers, for example, alcohol specifically such as ethanol, polyalcohol such as propylene glycol and polyethylene glycol, and nonionic surfactants such as polysorbate 80 (TM) and HCO-50.

Oily liquids include sesame seed oil and soybean oil, and benzyl benzoate and benzyl alcohol may be used with the oily liquids as solubilizers. Furthermore, buffers, for example, phosphate buffer and sodium acetate buffer; soothing agents, for example, procaine hydrochloride; stabilizers, for example, benzyl alcohol and phenol; and anti-oxidants may be mixed into the oily liquids. The prepared injections are generally filled in appropriate capsules.

The dose may vary, depending on the type and severity of disease; compound to be administered and the route to be administered; age, sex, body weight and such of patient, and the like. In case of oral dosing, generally, 0.1 to 1,000 mg, preferably 1 to 300 mg of compound A is administered per day to an adult in one dose or in divided portions.

Furthermore, the compound of the present invention is applicable as pharmaceutical agents for animals.

Pharmacological tests

[1] Therapeutic effect in allergic dermatitis model animals

Type I and Type IV allergic reactions are suggested to be complicated in atopic dermatitis. Thus, a model where Type I and Type IV allergic reactions occur singly or in combination is useful.

1. Effect on murine DNFB-induced allergic dermatitis

The model is produced by repeating antigen sensitization and induction in mouse to induce contact dermatitis that involves increase of IgE antibody titer, i.e., inflammation similar to atopic dermatitis (J. Allergy Clin. Immunol., 100 (6Pt2), 39-44, December 1997). In the model, inflammation is suggested to occur via the delayed type allergic reaction due to T cells and the late phase allergic reaction due to mast cells. Furthermore, the weight of the spleen was measured simultaneously in the test to examine systemic immunosuppressive action of a test compound.

Test methods

*Preparation of test compound

Preparation of solvent: Methyl cellulose (hereinafter referred to as MC) was dissolved in distilled water to prepare an aqueous 0.5% (w/v) MC solution.

Preparation of test compound: According to Examples 3 to 5 of JP-A 2000-256323, compound A was synthesized. A given amount of compound A was suspended in the above described solvent to prepare a 1 mg/mL suspension. Furthermore, by dilution, 0.1 mg/mL and 0.01 mg/mL suspensions were prepared. In addition, as positive controls, 0.5 mg/mL, 0.2 mg/mL and 0.1 mg/mL solutions of prednisolone (Sigma) were similarly prepared. Prednisolone is one of the adrenocorticosteroids that are suggested to be effective for the treatment of atopic dermatitis.

*Preparation and application of antigen

Antigen preparation: 0.15% (w/v) solution of DNFB (2,4-dinitrofluorobenzene) was prepared in a mixed solution of acetone and olive oil (3:1, v/v) on needed basis.

Antigen application: 25 μL of the antigen solution was applied on each sides of both of the ears of a 9-week-old female BALB/c mouse (Japan SLC Inc.) once in a week for 5 times in total.

*Administration of test compound

During the period from the next day of the third antigen application to the next day of the fifth antigen application, 10 mL/kg of the above-described test compound was administered once a day 15 times in total. Herein, on the day of antigen application, the test compound was administered one hour before the antigen application while the test compound was administered 23 hours after the antigen application on the next day of the antigen application.

*Measurement of ear swelling

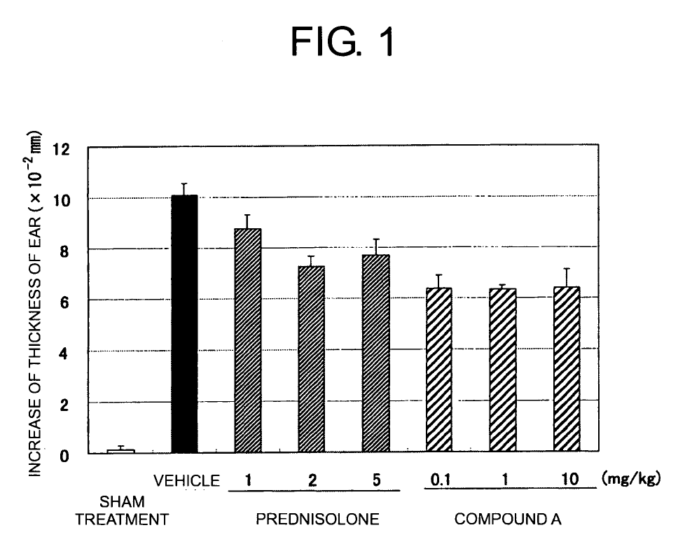

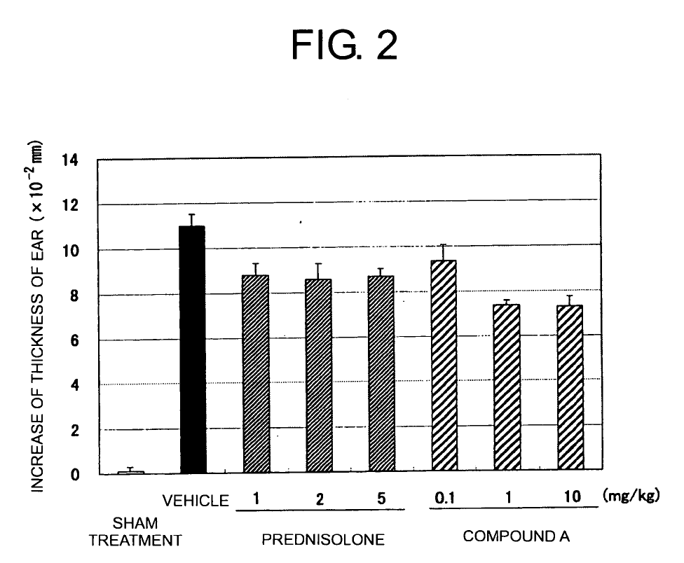

Before and 24 hours after the antigen application, the thickness of the ears was measured with a dial thickness gauge (Mitutoyo Corp.). The difference of thickness was used as an index of swelling. FIGS. 1 and 2 show the results of the measurement at the fourth and fifth antigen applications, together with the results of the positive controls.

*Measurement of spleen weight

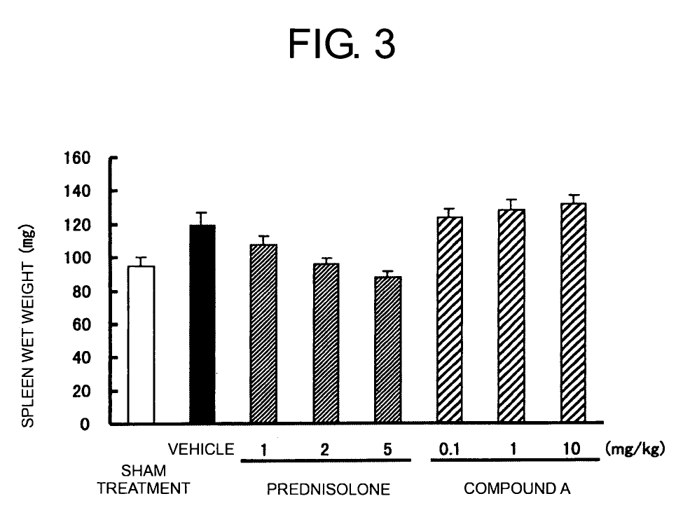

24 hours after the fifth antigen application, the spleen was harvested from the mouse exsanguinated under anesthesia with ether. The wet weight of the spleen was measured. The results are shown in FIG. 3.

*Results

Nagai et al. reported that ear swelling, complexed with the late phase response (Type I allergic reaction) and the delayed-type response (Type IV allergic reaction), develops after the fifth antigen application in this model.

Compound A significantly suppressed ear swelling in the allergic dermatitis model. Additionally, compound A showed effect at the start of dosing after the third antigen application. No decrease of spleen weight was observed for compound A as observed in the case of prednisolone.

2. Effect on murine IgE-dependent allergic dermatitis

A model was produced by passive sensitization of a mouse with IgE and repeated antigen challenge to trigger triphase (early phase, late phase and very late phase) dermatitis (Pharmacology, 60(2), 97-104, February 2000). Relation of mast cells and T cells, as well as invasion of eosinophils in local inflammatory sites is confirmed in these reactions. Thus, these reactions are suggested to reflect a part of the symptoms of atopic dermatitis.

Test methods

*Preparation of test compound

Preparation of solvent: MC was dissolved in distilled water to prepare an aqueous 0.5% MC solution.

Preparation of test compound: A given amount of compound A was suspended in the above-described solvent to prepare a 1 mg/mL suspension.

In addition, as positive controls, similarly to the test compound, 1 mg/mL ketotifen fumarate (Sigma) and 3 mg/mL pranlukast hydrate (extracted from Onon™ (Ono Pharmaceutical Co., Ltd.)) were prepared. Pranlukast hydrate is used as a leukotriene inhibitor for therapeutic agents for asthma and allergic rhinitis. Ketotifen fumarate is used as a chemical mediator release suppressor for asthma, allergic rhinitis, eczema, dermatitis, urticaria, dermal pruritis and allergic conjunctivitis.

*Passive sensitization Anti-DNP IgE (antibody against DNP; Yamasa Corp.) was prepared to 15 μg/mL with physiological saline. 0.2 mL of the resulting solution was administered via the caudal vein to a 9 week-old female BALB/c mouse (Japan SLC Inc.).

*Antigen preparation and application

Antigen preparation: 0.15% (w/v) solution of DNFB (2,4-dinitrofluorobenzene) was prepared in a mixed solution of acetone and olive oil (3:1, v/v) on needed basis.

Antigen application: 24 hours after the administration of the above-described anti-DNP IgE, 25 μL of the antigen solution was applied on each sides of both of the ears.

*Administration of test compound

From the antigen application day up to day 8 after the antigen application, 10 mL/kg test compound was orally administered once a day 9 times in total. To another group of mice, 10 mL/kg test compound was orally administered once a day 8 times in total from one day after the antigen application up to day 8 after the antigen application. To a further another group of mice, similarly, 10 mL/kg test compound was orally administered once a day 7, 5 or 3 times in total from two, four or six days after the antigen application up to day 8 after the antigen application. During the period from the antigen application day to the start of the dosing of the test compound, in place of the test compound, only the solvent was orally administered at 10 mL/kg once a day. The test compound was administered one hour before the antigen application on the day of antigen application and one hour before the measurement of the thickness of the ear on day 8 after the antigen application.

*Measurement of ear swelling

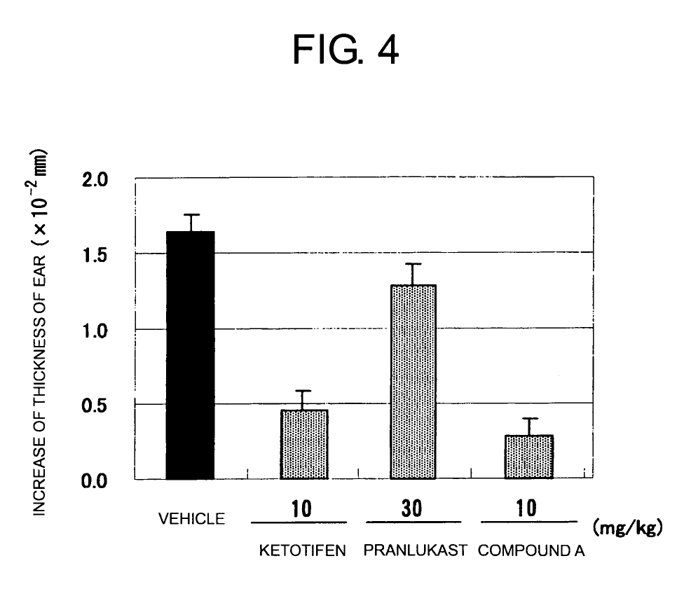

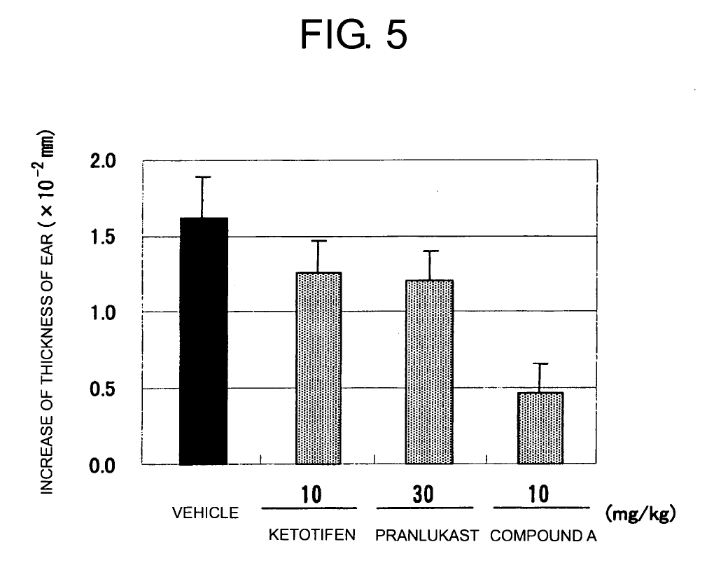

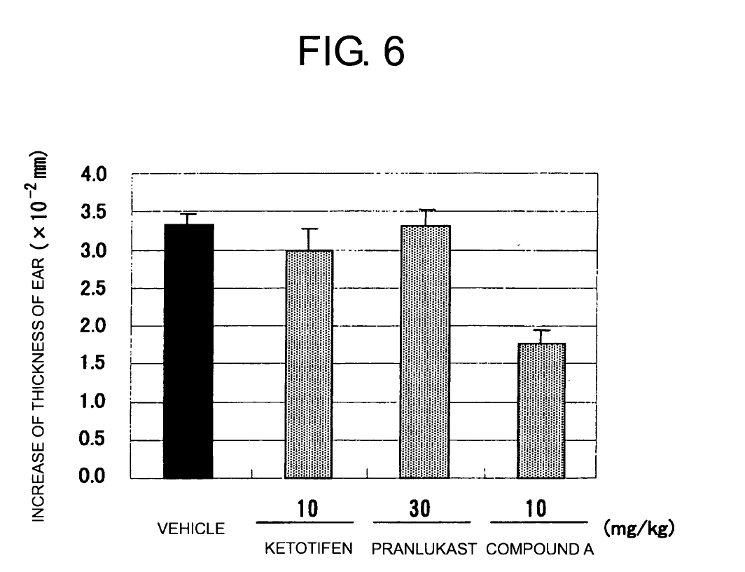

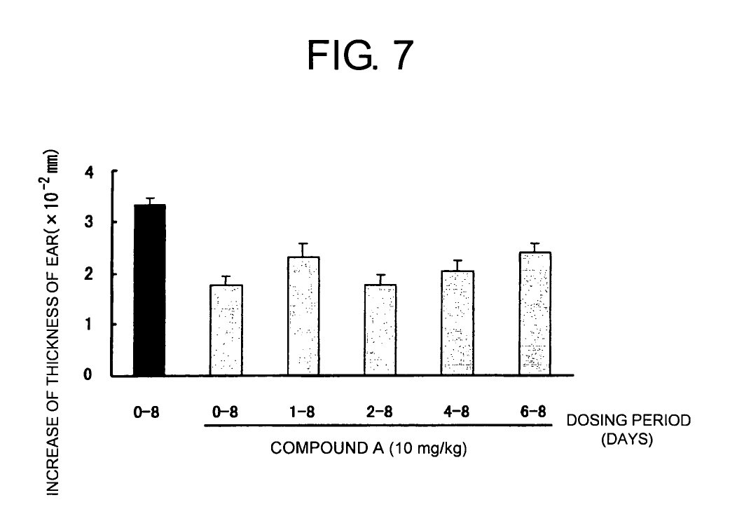

Before the antigen application and one hour, 24 hours and 8 days after the antigen application, the thickness of the ear was measured with a dial thickness gauge (Mitutoyo Corp.). The difference between the values measured before the antigen application and at each time point was used as the index of swelling. FIGS. 4 to 6 show the results of each measurement. Furthermore, FIG. 7 shows the influence of the timing of the compound dosing on the swelling-suppressing effect 8 days after the antigen application.

*Results

Compound A significantly suppressed ear swelling during any of the phases of early phase (one hour after application), late phase (24 hours after application) and very late phase (8 days after application) in the IgE-dependent dermatitis model. Furthermore, the effect of compound A in the very late phase was observed even in the case where the administration was started after the induction of the late phase.

[2] Therapeutic effect using asthma model

Effect on antigen-induced immediate type asthma, late-type asthma and airway hypersensitivity in guinea pigs

Test methods

*Preparation of test compound

A given amount of compound A was suspended in the aqueous 0.5% MC solution to obtain a solution of 60 mg/mL. The test compound was further diluted to 20, 6 and 2 mg/mL on needed basis. As positive controls, 6 mg/mL pranlukast hydrate (extracted from Onon™ (Ono Pharmaceutical Co., Ltd.)) and 6 mg/mL prednisolone (Sigma) were similarly prepared.

*Active sensitization and antigen challenge

Sensitization: Using an ultrasonic nebulizer (NE-U12; OMRON), a 6-weeks old male Hartley guinea pig (Kudo, Co., Ltd.) was continuously inhaled with 1% OVA (ovalbumin; Sigma)-containing physiological saline for 10 minutes per day for 8 consecutive days.

Antigen challenge: One week after the last sensitization, 2% OVA was similarly inhaled for 5 minutes. 24 hours before and one hour after the OVA challenge, metyrapone-containing physiological saline (Aldrich, 10 mg/mL) was administered intravenously and 30 minutes before the OVA induction, pyrilamine-containing physiological saline (Sigma, 10 mg/kg) was administered intraperitoneally.

*Administration of test compound

During the 15-days from the start of sensitization to the antigen challenge, 5 mL/kg test compound was orally administered once a day. During the 8 days of sensitization, the test compound was given one hour before the sensitization. On the day of the antigen challenge, the test compound was given one hour before the challenge. As solvent controls, the vehicle was similarly administered in accordance with OVA induction and physiological saline induction.

As positive controls, pranlukast hydrate was administered one hour before the challenge, while prednisolone was administered 16 hours and 2 hours before the challenge. The animals were fasted 16 to 18 hours before the oral administration.

*Measurement of airway resistance

Using total respiratory function analysis system (Pulmos-I, M.I.P.S. Company), the pre value was measured. Subsequently, specific airway resistance (hereinafter referred to as sRaw) per 100 breathes was measured one minute and 2, 4, 5, 6, 7 and 8 hours after the OVA challenge, and further once between 22 to 26 hours after the challenge, respectively. The average was used as sRaw at each measured time point. The increment ratio of sRaw was calculated by the following formula.

Increment ratio of sRaw (%)=[(sRaw at each measured time point −sRaw before the challenge)/(sRaw before the challenge)]×100

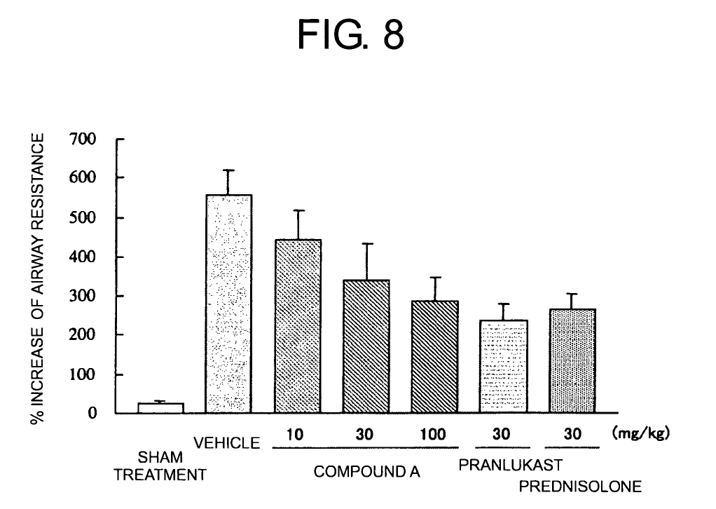

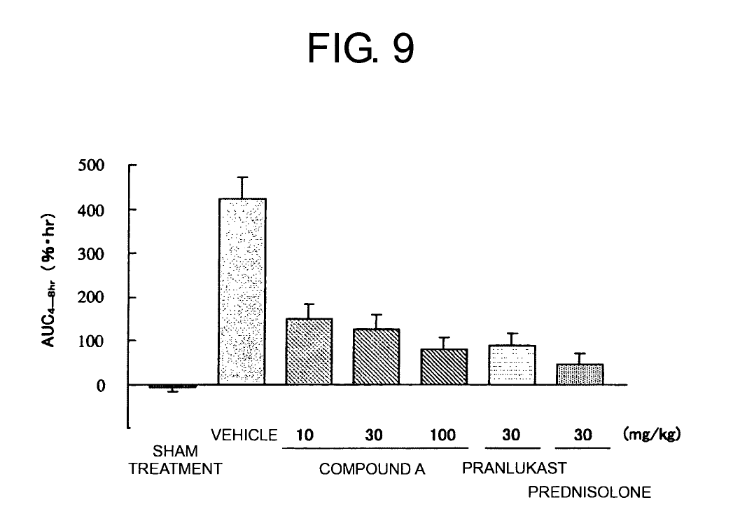

FIG. 8 shows the increment ratio of sRaw one minute after the OVA challenge and FIG. 9 shows the increment ratio of sRaw (area under the curve: AUC_{4-8 hr}) over 4 to 8 hours after the challenge.

*Measurement of airway reactivity

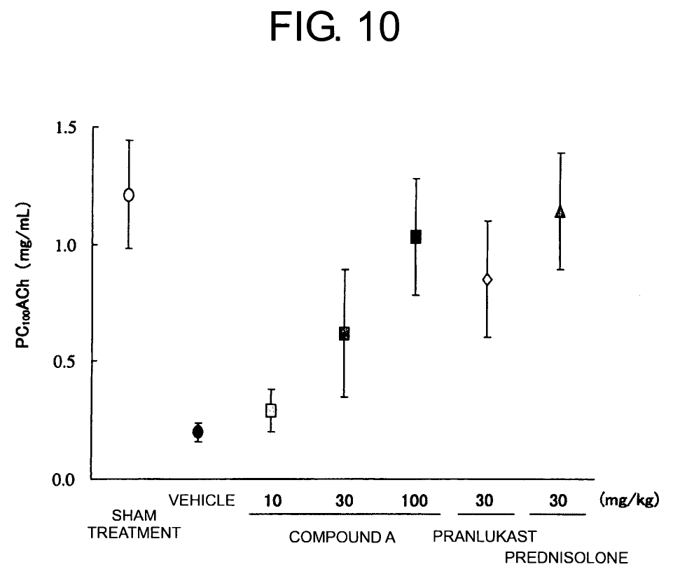

Twenty-two to 26 hours after the antigen challenge, each of 0.0625, 0.125, 0.25, 0.5, 1 and 2 mg/mL solutions of physiological saline and acetylcholine (hereinafter referred to as ACh) were sequentially inhaled for one minute, until the sRaw was 2-fold or more to the baseline sRaw (sRaw after inhalation of physiological saline). Based on the ACh concentration and the concentration-resistance curve of sRaw, the ACh concentration required for the sRaw to achieve 100% increase from the baseline sRaw, i.e., PC100ACh was determined. The measured results are shown in FIG. 10.

*Results

In this model, compound A suppressed all of the antigen-induced immediate type asthma response (sRaw immediately after antigen challenge), late asthmatic response (sRaw over 4 to 8 hours after antigen challenge) and airway hypersensitivity. The positive controls pranlukast hydrate and prednisolone also suppressed all of the antigen-induced immediate type asthma response, late-type asthma response and airway hypersensitivity.

[3] Effect on leukotriene production

Leukotrienes (hereinafter referred to as LTs) are known to be produced by basophils, mast cells and so on, and to be involved in the exacerbation of allergic disease, particularly allergic bronchial asthma.

1. Effect on leukotriene production from human basophils

*Preparation of test compound

A given amount of compound A was prepared in DMSO (dimethyl sulfoxide) to a concentration of 0.01 mM, and then diluted with Tyrode solution (Sigma) to prepare 100 μM to 0.1 μM compound A solutions (1% DMSO solution). To act them on cells, the solutions were further diluted to 10 μM to 0.01 μM (0.1% DMSO solution).

*High purification of basophils

Using a syringe charged with 3.8% sodium citrate solution, 100 mL blood was obtained from human blood.

Using 10× HBSS (−) (10×Hank's balanced salt solution, GIBCO), Percoll (Amersham) and Milli Q water, 1.070 g/mL, 1.079 g/mL and 1.088 g/mL Percoll-HBSS (−) were prepared and layered, and the obtained blood was loaded on the layers and centrifuged at 300× g for 25 minutes. A cell fraction between the 1.070 g/mL Percoll-HBSS (−) layer and the 1.079 g/mL Percoll-HBSS (−) layer was recovered. 3 volumes of HBSS (−) was added to the recovered cell suspension and centrifuged at 300× g at 4° C. for 7 minutes. After centrifugation, the supernatant was discarded and the cells were rinsed once with HBSS (−). Thus recovered cell population was used as the basophils.

*Preincubation

The basophils were prepared to 2.5×10⁶ cells/mL with Tyrode solution, and 10 μg/mL recombinant human IL-3 (Genzyme/Techne) was added to a final concentration of 100 ng/mL. Immediately thereafter, 80 μL/well (2.5×10⁵ cells/well) of basophils were seeded on a round-bottom 96-well plate and were incubated in 5% CO₂ at 37° C. for 30 minutes.

*Addition of test compound

After preincubation, 10 μL/well of the above-described test compound was added and incubated in 5% CO₂ at 37° C. for 10 minutes. 10 μL/well Tyrode solution containing 1% DMSO was added as the solvent control group.

*Addition of anti-human IgE antibody

10 μL/well of anti-human IgE antibody diluted with Tyrode solution to 1, 3, 10, 30 and 100 μg/mL was added and incubated in 5% CO₂ at 37° C. for 30 minutes (final concentrations were 0.1, 0.3, 1, 3 and 10 μg/mL, respectively).

*LTs Assay

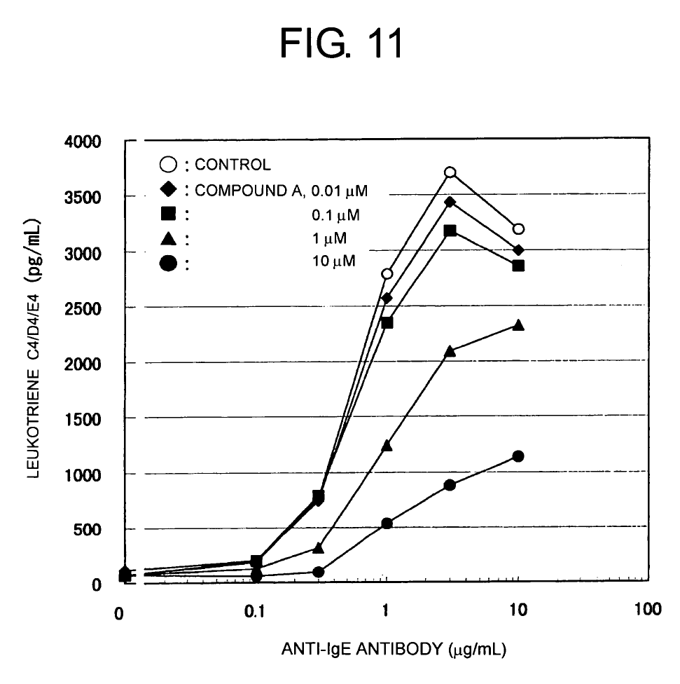

30 minutes after stimulation, the reaction mixtures were centrifuged at 3 000 rpm at 4° C. for 5 minutes to recover 80 μL/well supernatants. The LTs amount in the supernatants was assayed according to the manufacturer's protocol of LTs EIA kit (Amersham Pharmacia). The samples were diluted with Tyrode solution to 3-fold and 24-fold for the assay. The assay results are shown in FIG. 11.

*Results According to the test, compound A showed a suppressive action on the production of leukotrienes (C4/D4/E4) from human basophils

2. Effect on leukotriene production from rat mast cell line

*Preparation of test compound

A given amount of compound A was diluted and adjusted to 3, 1, 0.3 and 0.1 mM (100% DMSO solution). Then, the resulting solutions were diluted with E-MEM (EAGLE-MEM; Nikken Biomedical Lab.) to prepare solutions of 100 to 1 μM (1% DMSO solution). To act the solutions on cells, they were further diluted to 10 μM to 0.1 μM (0.1% DMSO solution).

*Preparation of PIPES Buffer

1 mM PIPES (Dojin Molecular Technologies, Inc.), 14 mM NaCl, 0.5 mM KCl, 0.06 mM MgCl₂, 0.1 mM CaCl₂, 0.55 mM glucose and 0.1% BSA (bovine serum albumin; Sigma) were prepared with purified water and then adjusted to pH 7.4 using NaOH.

*Preparation of anti-DNP IgE

1 mg/mL anti-DNP IgE (monoclonal murine anti-DNP IgE; Yamasa Corp.) was diluted to 1000 fold with the PIPES buffer to prepare a 1 μg/mL solution.

*Preparation of DNP-BSA

10 mg/mL DNP-BSA was diluted to a concentration of 10 μg/mL with the PIPES buffer.

*Method of culturing rat mast cell line

Culture medium: E-MEM containing heat-inactivated 10% FCS (fetal calf serum; Morgate Biotech), 100 units/mL penicillin and 100 μg/mL streptomycin (in the form of penicillin/streptomycin; GIBCO).

*Cell preparation

After centrifuging and washing rat mast cell line RBL-2H3 (Human Science; 1×10⁶cells/mL/tube) with the above-described culture medium, the cells were resuspended in the culture medium and cultured in a 75-cm² flask (Falcon 353136) for 3 days. After subculture, the cells were further cultured in a 225-cm² flask (Corning 431082) for 2 days. Semi-confluency (60 to 70% confluency) of the cells was confirmed and the cells were rinsed with HBSS and detached with trypsin-EDTA. After the cells were recovered, were washed by centrifugation with the culture medium, and then resuspended in the culture medium. The concentration of the cells was adjusted to 2×10⁵ cells/mL and were seeded at 250 μl/well on a 96-well flat bottom culture plate (Falcon 3072) and cultured in 5% CO₂ at 37° C. for 20 hours.

*Antigen sensitization

The culture medium was discarded from the plate, the cells were rinsed with HBSS, 100 μL/well of 150 ng/mL anti-DNP IgE was added and incubated at 37° C. for 30 minutes for cell sensitization.

*Addition of test compound

The culture medium was discarded from the plate, the cells were rinsed with HBSS, 80 μL/well culture medium was added followed by 10 μL/well compound A diluted with the culture medium to 1, 3, 10, 30 and 100 μM (the final concentrations were 0.1, 0.3, 1, 3 and 10 μM, respectively, in DMSO at a final concentration of 0.1%) and then incubated at 37° C. for 10 minutes.

*Antigen stimulation

10 μL/well DNP-BSA diluted with the culture medium to 150, 500, 1500 and 5000 ng/mL was added (final concentrations were 15, 50, 150 and 500 ng/mL, respectively) and incubated at 37° C. for 30 minutes.

*LTs assay

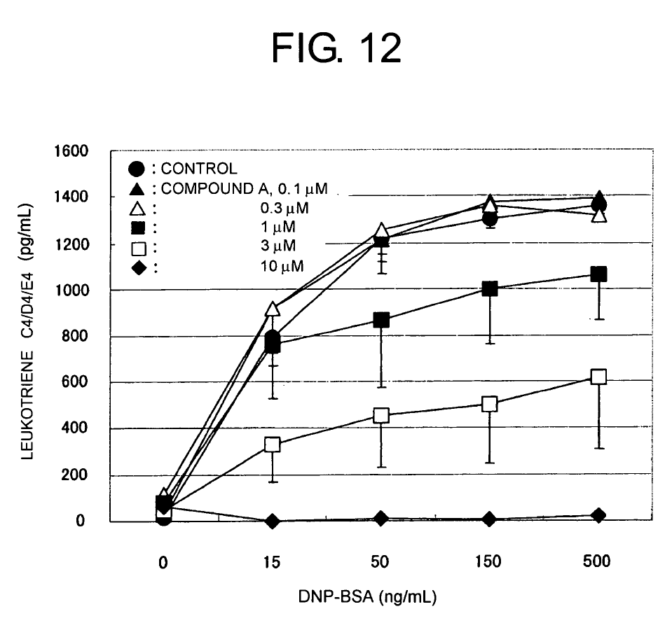

30 minutes after the antigen stimulation, 20 μL/well supernatant was recovered and the LTs amount was measured according to manufacturer's protocol of LTs EIA kit (Amersham Pharmacia). The assay results are shown in FIG. 12.

*Results

According to the present test, compound A exerted a suppressive effect on leukotriene production (C4/D4/E4) in the rat mast cell line.

[4] Cannabinoid receptor-binding assay

Compound A is known as a modulator selective for peripheral cell type cannabinoid receptor (IC₅₀ for CB1 and CB2 are 3436 nM and 0.087 nM, respectively) (Pharmacological test results, Table 33 and Examples 3-5 of JP-A 2000-256323).

3. Effect of CB2 inverse agonist and CB2 agonist on murine IgE-dependent allergic dermatitis reactions

Using a triphase dermatitis model that can be induced by antigen in mouse passively sensitizationed with IgE, the actions of CB2 inverse agonist and CB2 agonist were examined.

Test methods

*Animal: 8 to 10 weeks-old Female BALB/c mouse (Japan SLC Inc.) was used.

*Preparation of test compound

Preparation of solvent: MC was dissolved in distilled water to prepare an aqueous 0.5% MC solution.

Preparation of test compound: A given amount of compound A was suspended in the solvent to prepare 0.01, 0.1 and 1 mg/mL suspensions.

0.5 mg/mL prednisolone as a positive control, 1 and 5 mg/mL CB2 specific agonist, HU-380, and 0.01, 0.1 and 1 mg/mL CB2 specific inverse agonist, SR144528, as comparative controls were similarly prepared as MC suspensions as described above.

*Passive sensitization

Anti-DNP IgE (antibody against DNP; Yamasa Corp.) was prepared to 15 μg/mL with physiological saline. 0.2 mL of the resulting solution was given through the caudal vein to the mouse.

*Antigen preparation and application

Antigen preparation: 0.15% (w/v) of DNFB (2,4-dinitrofluorobenzene) was prepared in a mixed solution of acetone and olive oil (3:1, v/v) on needed basis.

Antigen application: 25 μl of the antigen solution was applied on each side of both of the ears 24 hours after the administration of the anti-DNP IgE.

*Administration of test compound

From the next day of the antigen application to day 8 after the antigen application, 10 mL/kg test compound was orally administered once a day 9 times in total. On the day of antigen application, the test compound was administered one hour before the antigen application and the test compound was administered one hour before the measurement of the thickness of the ear on day 8 after the antigen application.

*Measurement of ear swelling

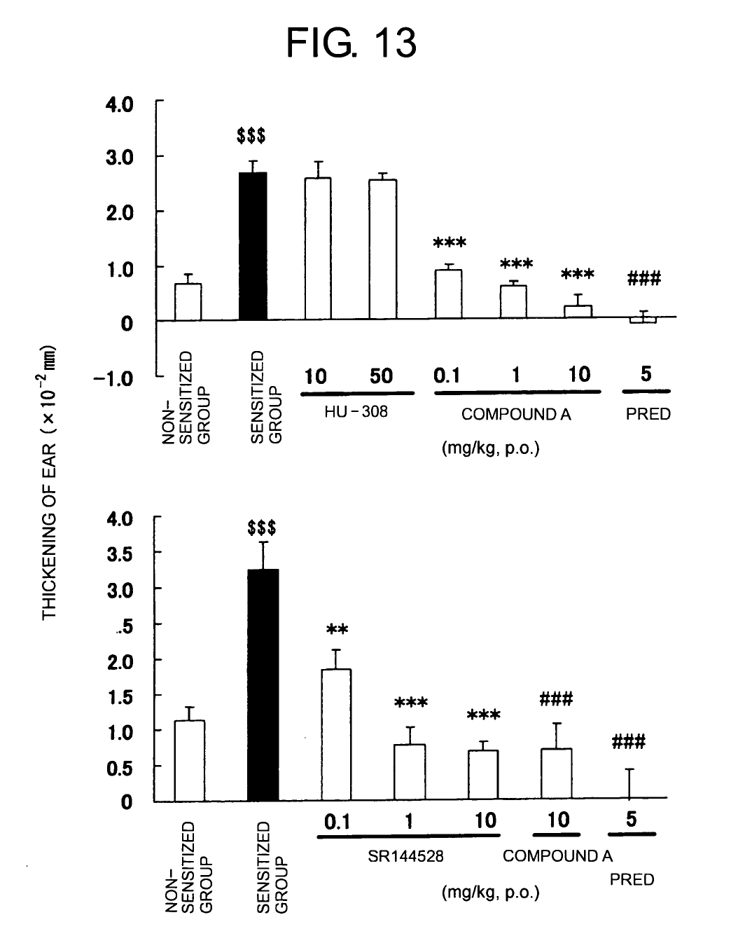

Before and 8 days after the antigen application, the thickness of the ear was measured with a dial thickness gauge (Mitutoyo Corp.). The difference between the value measured before the antigen application and at each time point was used as an index of swelling. FIG. 13 shows the results of the measurement.

*Measurement of organ weight

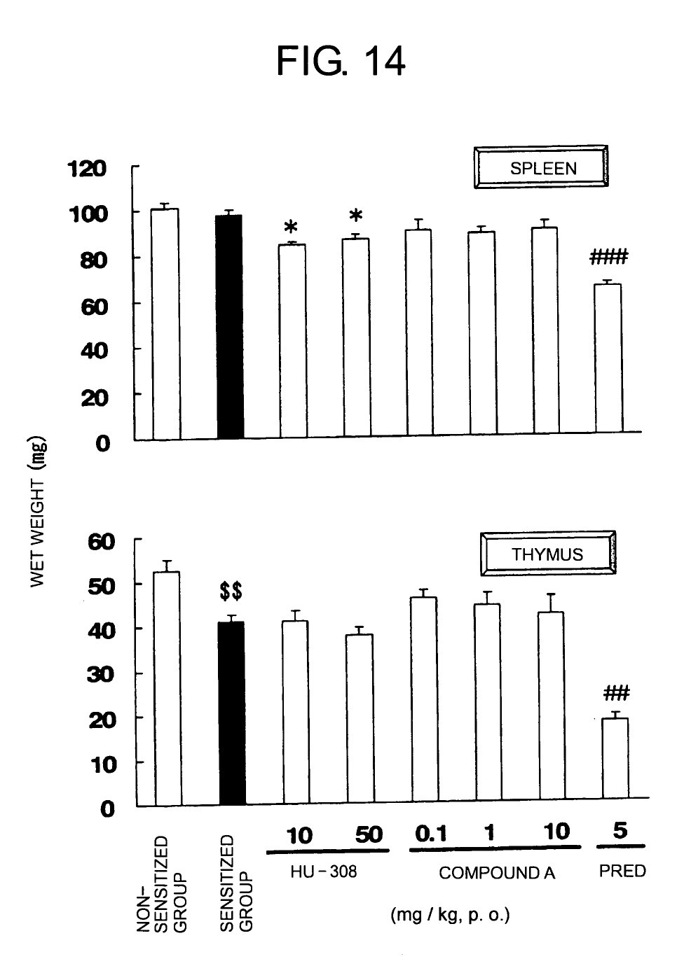

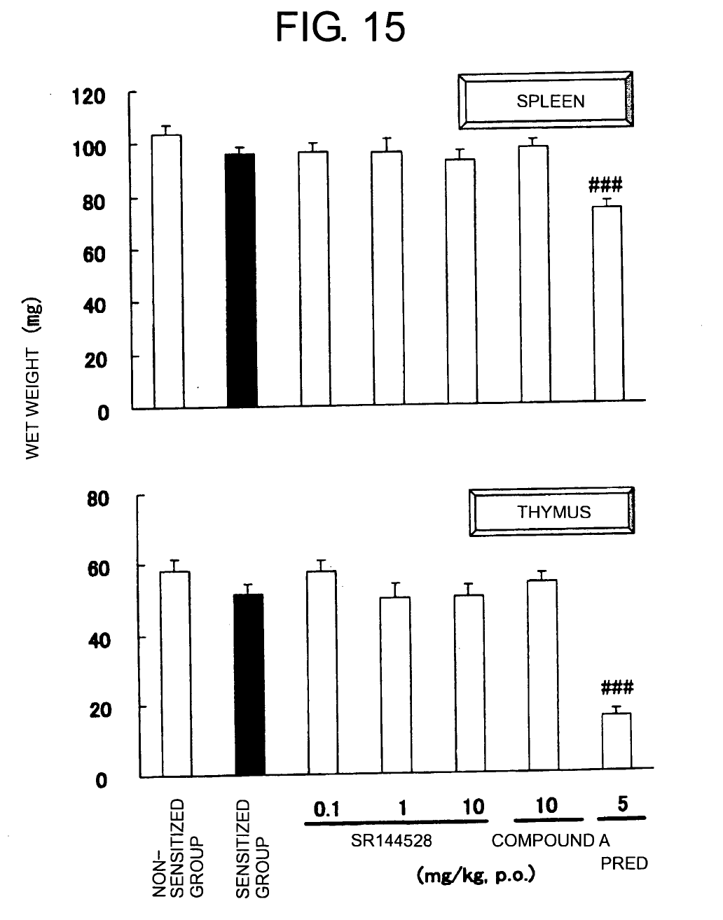

After the measurement of ear swelling, spleen and thymus were harvested to weigh their wet weights. The results of the measurement are shown in FIGS. 14 and 15.

*Results

Compound A significantly suppressed ear swelling at all the doses of 0.1, land 10 mg/kg at the very late phase (8 days after application). The CB2 inverse agonist SR144528 also showed significant effect at a concentration of 0.1 mg/kg or higher. On the other hand, no pharmacological effect was observed for the CB2 agonist HU-308 at any of the concentrations between 10 and 50 mg/kg. According to the result of weighing the spleen and thymus, in comparison to the significant suppression of the weight of both of the organs by prednisolone, no apparent changes could be observed by compound A and SR144528. Significant decrease in the weight of the spleen was observed for animals that were given HU-308.

4. Ear swelling induced by CB2 agonist and effect of compound A

CB2 inverse agonist showed efficacy in the IgE-dependent allergic dermatitis model. Therefore, 2-arachidonoylglycerol ether (2-AG-E), the stable form of an endogenous ligand candidate 2-arachidonoylglycerol, and a specific CB2 agonist HU-308 were examined whether they directly induce ear swelling and compared to the ear swelling induced by arachidonic acid (AA). Furthermore, the action of compound A on the influence of CB2 agonist on ear was examined.

Test methods

*Animal: 8 to 10 weeks-old female BALB/c mouse (Japan SLC Inc.) was used.

*Preparation and application of test substances

Synthetically produced 2-AG-E and HU-308 were prepared with acetone to 1 or 10% (w/v) and 10% (w/v), respectively, and AA (Sigma) was prepared with acetone to 1.25% (w/v), on needed basis. 10 μL each of these substances was applied on each side of the left ear.

*Preparation of solvent and compound A

Preparation of solvent: MC was dissolved in distilled water to prepare an aqueous 0.5% MC solution.

Preparation of compound A: A given amount of compound A was suspended in the solvent to prepare 0.001, 0.01, 0.1 and 1 mg/mL suspensions.

*Administration of compound A

10 mL/kg of the solvent or compound A was orally administered. One hour later, 10 μL each of 10% (w/v) 2-AG-E was applied on each side of the left ear.

*Measurement of ear swelling

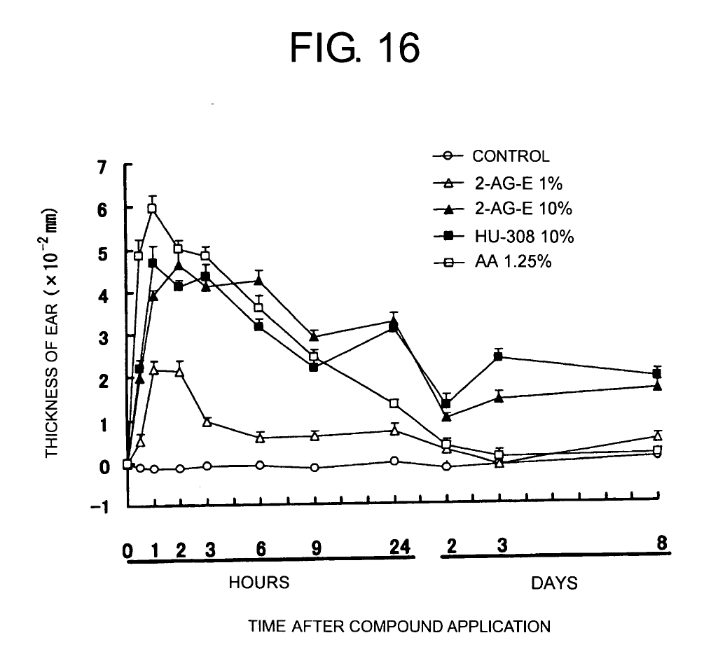

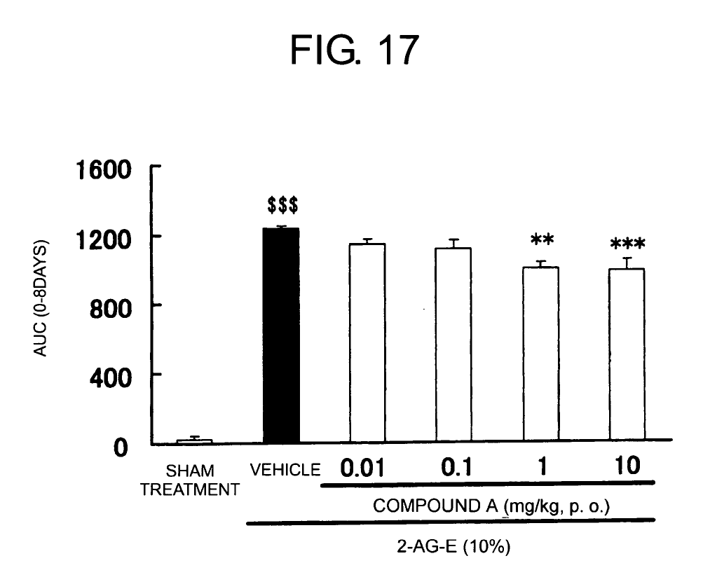

Before the application of the test substance, and one, 2, 3, 6, 9 and 24 hours, and 2, 3 and 8 days after the application, the thickness of the ear was measured with a dial thickness gauge (Mitutoyo Corp.). The difference between the value measured before the antigen application and the value at each time point was used as an index of swelling. For the evaluation of compound A, the area under the curve obtained from the change of ear swelling over time up to day 8 after 2-AG-E application was calculated and used. The results of individual measurements are shown in FIGS. 16 and 17.

*Results

2-AG-E application resulted in a concentration-dependent ear swelling that reached a peak in 1 to 2 hours after the application (early phase). Subsequently, continuous swelling was detected which corresponds to the late phase and the very late phase after 24 hours. HU-308 also induced a similar continuous ear swelling. On the other hand, AA induced a swelling that reached a peak one hour after the application which level was comparable to that induced by 10% 2-AG-E, but the swelling disappeared after 2 days.

In a dose-dependent manner, compound A suppressed ear swelling induced by the application of 10% 2-AG-E. 1 or 10 mg/kg of compound A gave a significant effect.

Thus, CB agonist HU-308 and endogenous CB agonist 2-AG-E (ether form of 2-AG which is assumed to have a similar effect to 2-AG) induced dermatitis in animal models. Furthermore, compared to AA that exhibit no CB-binding activity, the symptoms of dermatitis induced by HU-380 and 2-AG-E were severe in the late phase and the very late phase.

Furthermore, compound A that is a CB2 inverse agonist suppressed inflammation caused by allergy in a dose-dependent manner.

These findings suggest that CB2 inverse agonists can be used for the treatment of diseases caused by CB agonists.

The fact that CB2 inverse agonists showed an effect comparable to or stronger than those exhibited by steroidal compounds and immunosuppressants that are apprehended to have side effects, supports that the CB2 inverse agonist may be used as a safer drug.

5. Effect on spontaneous scratching reaction of NC mouse

Itching is one of the main symptoms in the field of dermatology encompassing atopic dermatitis, urticaria and contact dermatitis. However, most of the mechanisms of the onset of itching has not yet been elucidated and no pharmaceutical agent that dramatically suppresses itching and has less side effect has been developed.

Currently, NC mouse is used as an animal model of atopic dermatitis. No dermatitis or scratching action is observed when the mouse is kept in an environment under the control of atmospheric microorganisms (in SPF environment). However, when the mouse is kept in a conventional environment, scratching action together with the onset of dermatitis can be observed from about week 8 and the symptom progresses into chronic symptom (J. Dermatol. Sci., 25, 20-28, 2001).

Test methods

*Preparation of test compound

Preparation of solvent: MC was dissolved in tap water to prepare an aqueous 0.5% (w/v) MC solution.

Preparation of test compound: A given amount of compound A was suspended in the above-described solvent to prepare 1 mg/mL and 0.1 mg/mL suspensions. As positive controls, betamethasone valerate (Sigma) and tacrolimus hydrate (extracted from Prograf (Fujisawa Pharmaceutical Co., Ltd.)) were similarly prepared to 1 mg/mL. Betamethasone valerate is one of the adrenocorticosteroids that is considered to be effective for the treatment of atopic dermatitis. Tacrolimus hydrate is a therapeutic agent of atopic dermatitis which is known as an immunosuppressor as described above.

*Breeding of animal and screening method

Four weeks-old male NC/Jic mice (CLEA JAPAN, Inc.) were kept in the same cage for 12 days with mice (A) infected with rodent mite (Myoba musculi) that exhibit severe dermal lesions. The mice (A) were taken away from the cage and the NC/Jic mice were used at the age of 16 weeks.

Breeding conditions: The mice were fed with solid feed CA-1 (CLEA JAPAN INC.) ad libitum and tap water as drinking water ad libitum, and kept at a temperature of 22±2° C. and a humidity of 55±10% under lighting from 8:00 a.m. to 20:00 p.m.

From 10 days before the start of the experiment, the number of scratching behavior with hind legs of the mice was visually counted (for 20 minutes; once daily) over 2 days or 3 days. Among the plural mice counted, mice with 50 or more average scratching movements per day were selected for use.

*Administration of test compound

10 mL/kg test compound was orally administered once a day for 3 weeks.

*Test method

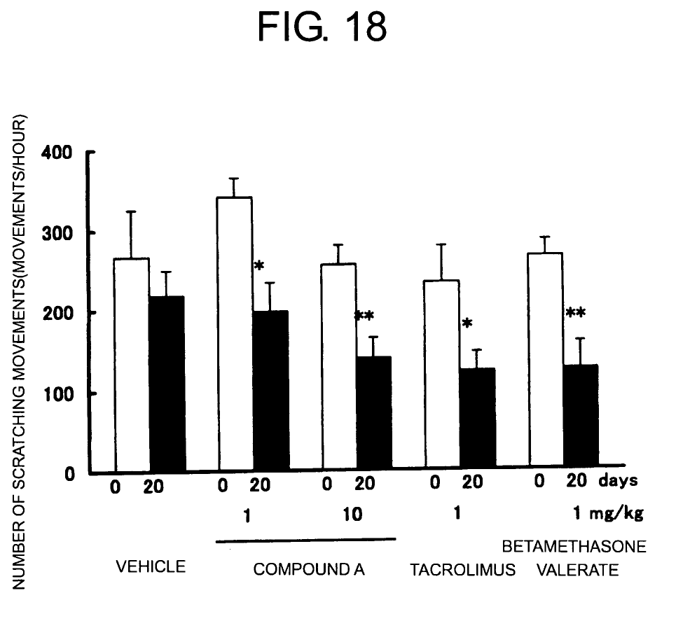

The behavior of the mice was filmed under unattended environment with a video camera to count the scratching motion with hind legs for one hour. Generally, the mice showed several scratching motions for about one second. This series of scratching motions was counted as one scratching movement, and all such scratching movements were counted irrespective of scratched sites. The measurement was done on the starting day of administration, and one day, 3, 6, 10, 13, 17 and 20 days after the start of administration. The results are shown together with the results of the positive controls in FIG. 18.

*Results

Compared with the control administered with the solvent alone, compound A suppressed the number of scratching movements in the scratching reaction model. The positive controls, tacrolimus hydrate and betamethasone valerate, also suppressed the number of scratching movements.

6. Action of compounds on cAMP production

The influence of various compounds on cAMP production in forskolin-stimulated hCB2-expressing CHO cells was studied by a similar method as described in The Journal of Pharmacology and Experimental Therapeutics, vol. 296, No. 2, pp. 420-425 (2001) (non-patent document 3). The measured results are shown in Table 1.

*Results

TABLE 1

| | Amount of added | Amount of cAMP production |
|---|---|---|
| Forskolin (5 μM) + compound | compound (μM) | compared to control (ratio (%)) |
| 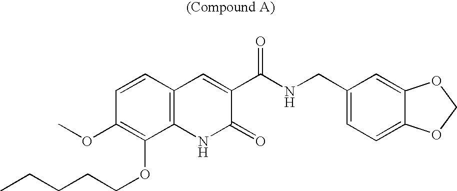 | 0.1 | 430 |
| 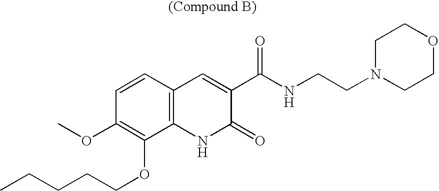 | 0.1 | 430 |
| 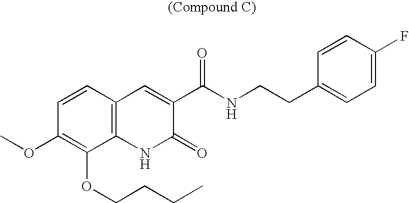 | 0.1 | 212 |
| 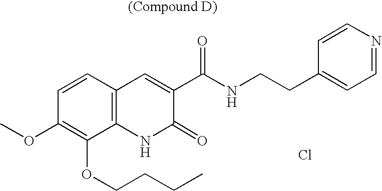 | 0.1 | 208 |
| 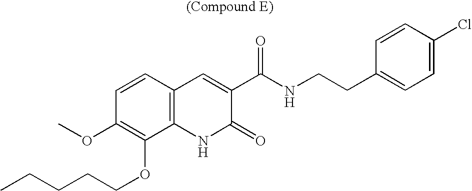 | 0.1 | 518 |
| 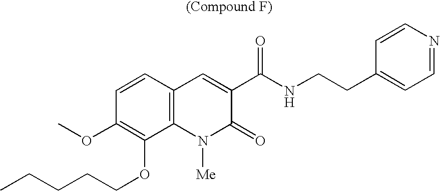 | 0.1 | 218 |
| 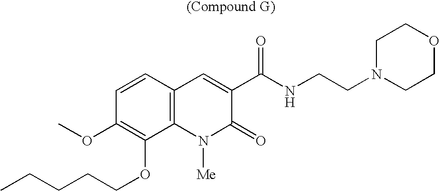 | 0.1 | 425 |
| 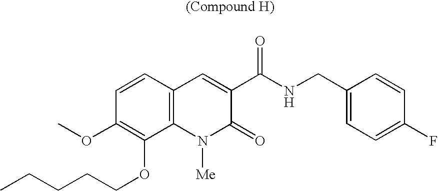 | 0.1 | 236 |
| 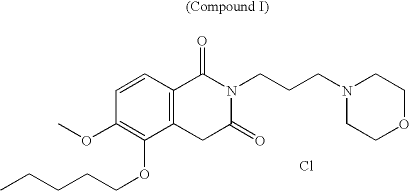 | 0.1 | 125 |
| WIN55212-2 | 0.01 | 4.3 |

Compounds A to I were revealed to be CB2 inverse agonists, and WIN55212-2 and 2-AG-E were confirmed to be agonists.

In addition, compound A was found to suppress the agonistic action of 2-AG-E under the coexistence of 2-AG-E and compound A in the present test. These findings suggest that CB2 inverse agonists antagonize CB2 agonists, such as 2-AG or 2-AG-E, via CB2.

7. IgE-dependent ear swelling in CB2-deficient mice

CB2-deficient mice where tested whether the same allergic symptoms as found in wild-type mice can be induced in these mice.

Methods

*Animals

Female CB2-deficient (CB2-KO) mice and female wild-type (WT) mice (C57BL/6 J strain) were used as experimental animals (provided from Professor A. Zimmer, the Laboratory of Molecular Neurobiology, Department of Psychiatry, University of Bonn Medical School; Europ. J. Pharmacol., 396, 141-149, 2000).

*Antigen application

On the first day of experiment, 0.2 ml of anti-DNP IgE (5 μg/mL in physiological saline, Yamasa Corp.) was administered to the tail vein of each mouse for immunization. After 24 hours, 25 μL of freshly prepared solution of 0.15% (weight/volume) antigen (DNFB (2,4-dinitrofluorobenzene; Nacalai Tesque)) dissolved in a mixture of acetone and olive oil (3:1, vol/vol) were applied onto both sides of the right ears.

*Measurement of ear swelling

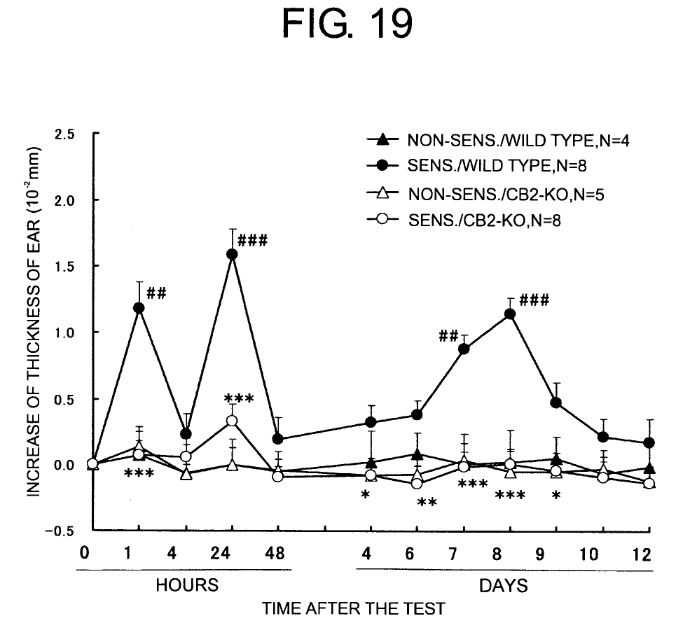

The swelling of the ear was measured over time before and after antigen application using a dial thickness gauge (Mitutoyo Corp.). The difference between the values measured before and after antigen application was defined as the degree of ear swelling and given as mean ± standard error (S.E.). Student's t-test was used in the statistical analysis and a significance level less than 5% was assumed to be significant. The test results are shown in FIG. 19.

*Results

Although wild-type mice developed triphasic (early phase, late phase and very late phase) dermatitis, CB2-deficient mice developed no marked dermatitis. Specifically, in the very late phase, the CB2-deficient mice developed no dermatitis which is a significant difference between the wild-type mice and the CB2-deficient mice.

This finding suggests that CB2 is closely associated with the triphasic symptoms of allergy, particularly, allergic symptoms in the late phase and very late phase. Specifically, the test results support that agents that comprise a CB2 antagonist or inverse agonist as an active ingredient serve as therapeutic agents for non-immediate-type allergic diseases, for example, atopic dermatitis.

8. 2-AG-E-induced ear swelling in CB2-deficient mice

It was tested whether allergic reaction can be induced in CB2-deficient mice using 2-AG-E (a reduced form of 2-AG and is predicted to exhibit a similar effect to 2-AG) that is considered to be an endogenous CB agonist.

Methods

*Animals

As described under the item of “7. IgE-dependent ear swelling in CB2-deficient mice”, female CB2-KO mice and female WT mice were used as experimental animals.

*Antigen application

10 μL of 2-AG-E (100 mg/mL acetone) was applied on each side of the left ears of the mice on the first day of experiment.

*Measurement of ear swelling

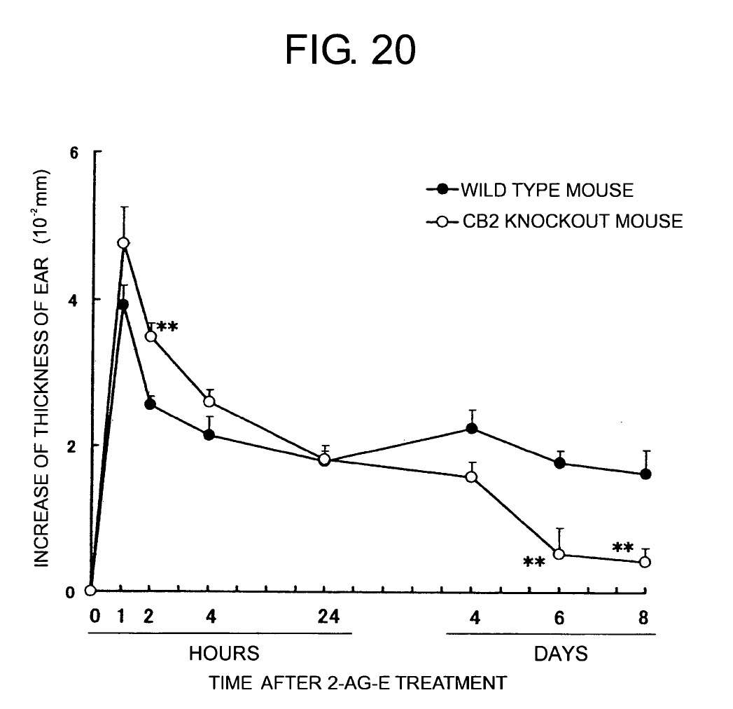

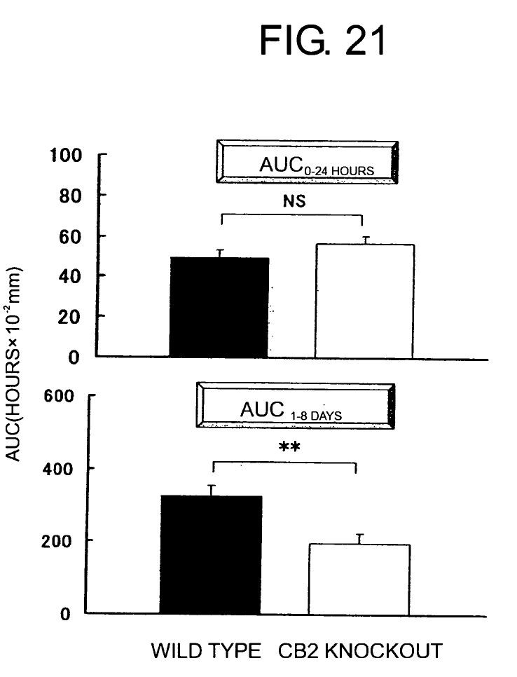

The swelling of the ear was measured over time before and after the application of 2-AG-E using a dial thickness gauge. The difference between the values measured before and after the antigen application was defined as the degree of ear swelling. Then, AUC (area under the curve: the degree of ear swelling ×time) was determined from the changes of the degree of ear swelling with the time course, and given as mean ± standard error (S.E.). Student's t-test was used in the statistical treatment. A significance level less than 5% was assumed to be significant. The test results are shown in FIGS. 20 and 21.

*Results

The wild-type mice developed dermatitis in the very late phase in addition to the early phase and late phase. On the other hand, the CB2-deficient mice developed dermatitis in the early phase, but the inflammation was milder in the late phase than that shown in wild-type mice and no significant inflammation was observed in the very late phase.

These findings show that among several known endogenous agonists, at least 2-AG and 2-AG-E are closely associated with allergic symptoms via CB2, and exhibit, in particular, great effect in the late phase and very late phase. Thus, the test results support that antagonists or inverse agonists that suppress the agonistic action of 2-AG and 2-AG-E serve as therapeutic agents for non-immediate-type allergic diseases, for example, atopic dermatitis.

9. 2-AG-E-induced ear swelling in mast cell-deficient mice

The degree of allergic symptoms was examined in mice deficient (partially deficient) of mast cells, which cells are considered to largely contribute to allergic reactions.

Methods

*Animals

Female mice of WBB6F1-+/+ (normal mouse) strain and WBB6F1-W/W^{v} (mast cell-deficient mouse) strain were purchased from Japan SLC, Inc. and used as experimental animals.

*Antigen application

10 μL 2-AG-E (100 mg/mL acetone) was applied on each side of the left ears of the mice on the first day of experiment.

*Measurement of ear swelling

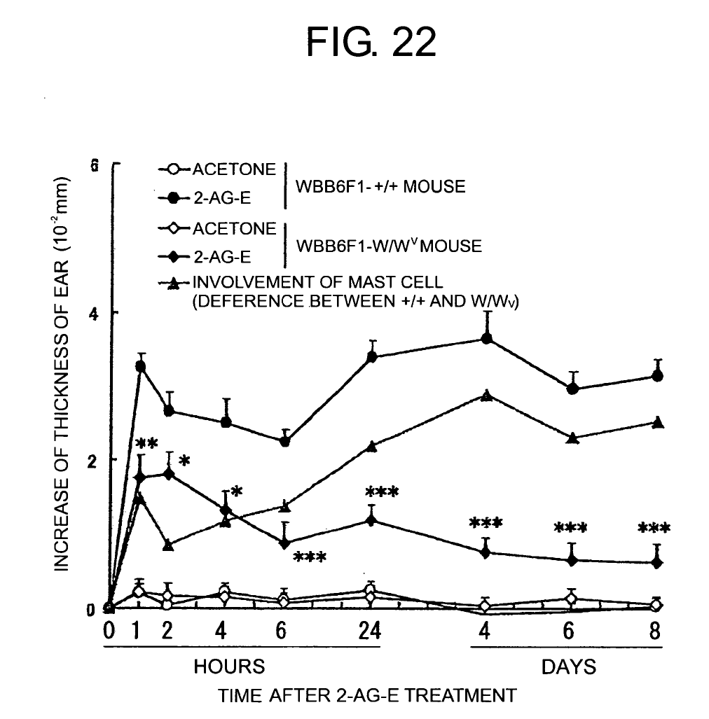

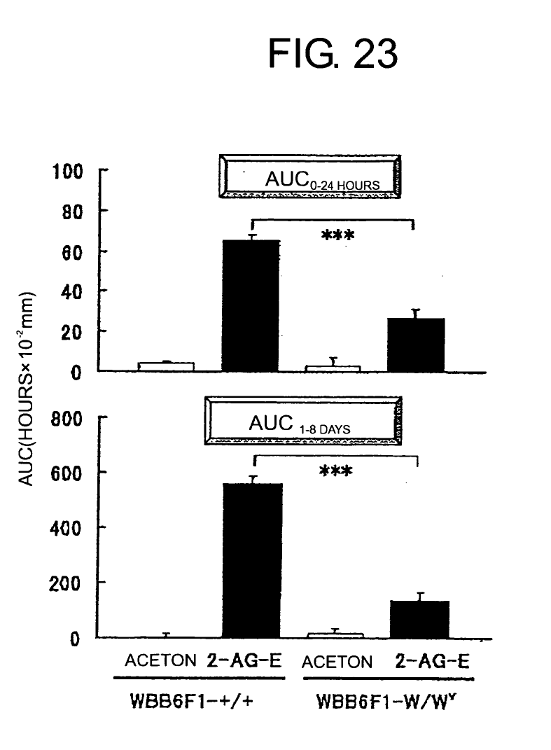

The swelling of the ear was measured over time before and after the application of 2-AG-E using a dial thickness gauge. The difference between the values measured before and after the antigen application was defined as the degree of ear swelling. Then, AUC was determined from the changes in the degree of ear swelling with the time course and given as mean ± standard error (S.E.). Student's t-test was used in the statistical treatment. A significance level less than 5% was assumed to be significant. The test results are shown in FIGS. 22 and 23.

*Results

Both of the wild-type mice and mast cell-deficient mice developed dermatitis, but the severity was quite different between the two groups. The wild-type mice developed more severe dermatitis. Furthermore, the severity of inflammation was particularly different between the two groups in the late phase and in the very late phase, than in the early phase.

These findings suggest the relation of 2-AG and 2-AG-E to mast cells in allergic symptoms, particularly in the late and very late phases. Thus, endogenous agonists, 2-AG and 2-AG-E, were revealed to be closely associated with the contribution of mast cells in allergic reactions. Accordingly, these findings also support that antagonists or inverse agonists that suppress the agonistic action of 2-AG and 2-AG-E serve as therapeutic agents for non-immediate allergic diseases, for example, atopic dermatitis.

Based on these results, an inverse agonist selective to the peripheral cell type cannabinoid receptor (CB2) was confirmed to be effective as a therapeutic agent for allergic diseases.

Particularly, the inverse agonists were effective in the treatment of non-immediate-type allergic diseases, for example, asthma and atopic dermatitis wherein immediate-type, late and delayed-type allergic reactions occur in combination. Furthermore, the effect of the inverse agonists to suppress allergic dermatitis in the late phase and very late phase is expected to be effective for the treatment of chronic dermatitis. In addition, the CB2-selective inverse agonist is expected to be effective for treating intractable allergic dermatitis, particularly atopic dermatitis, for which so far only steroidal agents and immunosuppressant tacrolimus hydrate are sufficiently effective.

Furthermore, the inverse agonists were found to be effective as anti-asthmatic agents that eases any of the symptoms of allergic asthma including antigen-induced immediate-type asthma, late-type asthma and airway hypersensitivity, and thus are expected to be effective for treating intractable asthma.

In addition, CB2-selective inverse agonists were revealed to reduce scratching behaviors that seem to be caused in response to allergic reaction in mouse itch-scratch response test. Hence, the CB2-selective inverse agonists are considered to be effective as antipruritic agents for treating pruritus associated with allergic diseases.

Furthermore, CB2-selective inverse agonists can be used as safe pharmaceutical agents that cause no systemic immune suppression, and may be used as oral drugs.

Compound A and SR144528 are known as CB2-selective inverse agonists. The CB2-selective agonist HU-308 and non-selective CB agonist 2-AG-E were revealed to exhibit no anti-allergic action, but instead, 2-AG-E induced allergic reaction. Furthermore, compound A was found to suppress the 2-AG-E-induced allergic reaction. These results obtained by the tests described above directly prove the usefulness of CB2-selective inverse agonists as anti-allergic agents.

In addition, the finding that CB2-deficient mice developed no dermatitis in the late phase and very late phase in the test of allergic reaction verifies the relation of CB2 to allergy and that agents which comprise a CB2 antagonist or inverse agonist as an active ingredient serve as therapeutic agents for non-immediate-type allergic diseases.

The finding that 2-AG-E is closely involved in allergic symptoms via CB2 indicated by the test of allergic reaction using CB2-deficient mice supports that antagonists or inverse agonists that suppress the agonistic action of 2-AG and 2-AG-E serve as therapeutic agents for non-immediate-type allergic diseases.

The result obtained by the test of allergic reaction using mast cell-deficient mice also supports the close relation of mast cells, 2-AG and 2-AG-E, and CB2, and that CB2 antagonists or inverse agonists serve as therapeutic agents for non-immediate-type allergic diseases.

Thus, the therapeutic effect on non-immediate-type allergic diseases of compound A, SR144528 and such can be attributed to the action of CB2. Thus, these compounds may be particularly effective as pharmaceutical agents that has a mode of action different from those of existing therapeutic agents for allergic diseases, for example, to symptoms that are resistant to existing therapeutic agents. In addition, it was suggested that the leukotriene-inhibiting effect of compound A potentiates its therapeutic effect.

Compounds A to H, compound I and SR144528, whose features with respect to their chemical structures differ to another, share a common pharmacological property as the CB2-selective inverse agonist. These facts revealed herein support the fact that CB2-selective inverse agonists are useful as therapeutic agents for allergic diseases.

In addition, based on the interaction of CB2 and 2-AG or 2-AG-E, CB2 antagonists or CB2 inverse agonists are expected to have therapeutic and preventive effects on diseases, other than non-immediate-type allergic diseases, that are related to 2-AG or 2-AG-E.

What is claimed is:

1. A therapeutic agent for a non-immediate-type allergic disease, which comprises as an active ingredient an inverse agonist of the peripheral cell type cannabinoid receptor.

2. The therapeutic agent for a non-immediate-type allergic disease according to claim 1, wherein the inverse agonist is a compound that exhibits the inverse agonistic action by antagonizing the agonistic action of 2-arachidonoylglycerol (2-AG) and/or 2-arachidonoylglycerol ether (2-AG-E).

3. The therapeutic agent for a non-immediate-type allergic disease according to claim 1, wherein the inverse agonist is a compound selected from the group consisting of: compound A, compound B, compound C, compound D, compound E, compound F, compound G, compound H, compound I and SR144528 shown below, and pharmaceutically acceptable salts thereof:

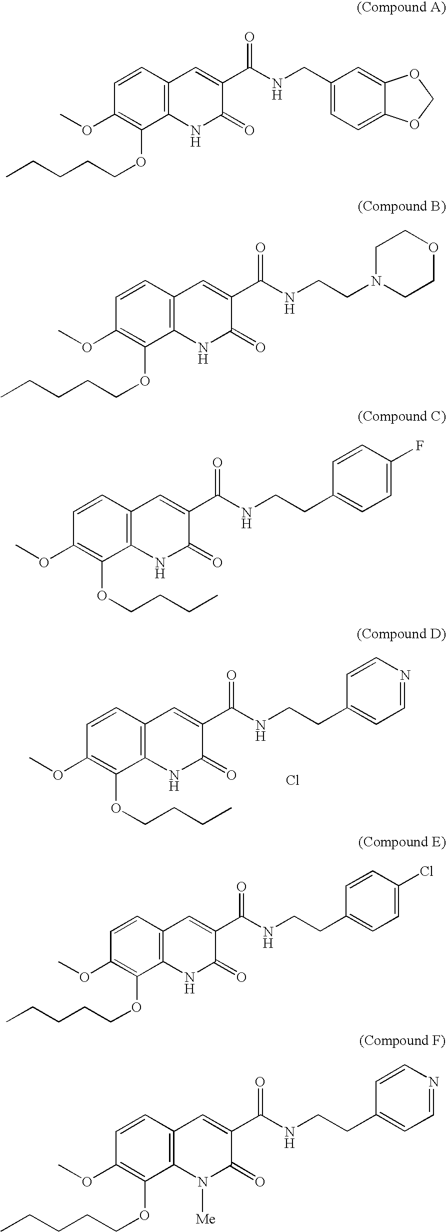

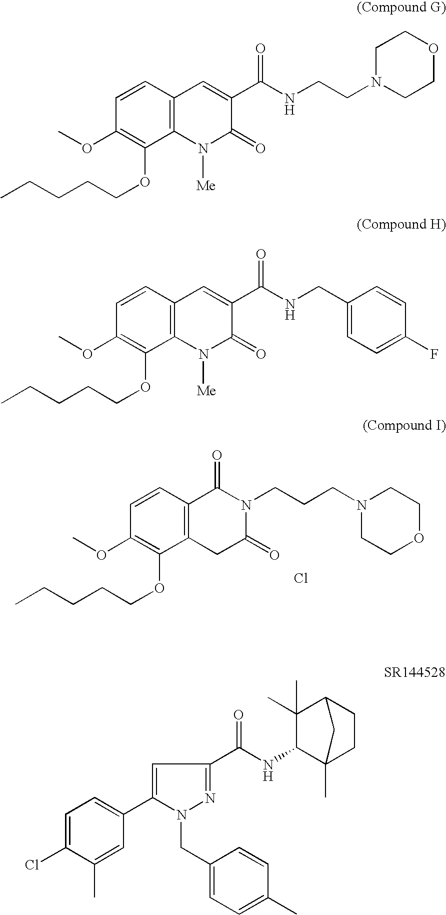

4. The therapeutic agent for a non-immediate-type allergic disease according to claim 1, wherein the non-immediate-type allergic disease is allergic dermatitis, allergic asthma, allergic rhinitis and/or allergic conjunctivitis.

5. The therapeutic agent for a non-immediate-type allergic disease according to claim 4, wherein the non-immediate-type allergic disease is allergic dermatitis.

6. The therapeutic agent for anon-immediate-type allergic disease according to claim 4, wherein the non-immediate-type allergic disease is allergic asthma.

7. The therapeutic agent for anon-immediate-type allergic disease according to claim 6, wherein the allergic asthma is a late asthmatic response and/or airway hypersensitivity.

8. The therapeutic agent for a non-immediate-type allergic disease according to claim 1, wherein the non-immediate-type allergic disease is a disease with late phase allergic reaction and/or delayed-type allergic reaction.

9. The therapeutic agent for anon-immediate-type allergic disease according to claim 1, wherein the inverse agonist of the peripheral cell type cannabinoid receptor is a compound that also has a leukotriene-inhibiting effect.

10. The therapeutic agent for a non-immediate-type allergic disease according to claim 1, wherein the non-immediate-type allergic disease is a disease associated with 2-AG and/or 2-AG-E.

11. A method for identifying a candidate compound for a therapeutic agent for a non-immediate-type allergic disease, which comprises the steps of:
(a) contacting a test compound with a cannabinoid receptor and an endogenous cannabinoid;
(b) determining the binding activity of the cannabinoid receptor to the endogenous cannabinoid; and
(c) selecting the compound that decreases the binding activity determined in step (b) compared with the activity determined in the absence of the test compound.

12. The method according to claim 11, wherein the cannabinoid receptor is CB2 and the endogenous cannabinoid is 2-AG or 2-AG-E.

13. A method for identifying a candidate compound for a therapeutic agent for a non-immediate-type allergic disease, which comprises the steps of:
(a) selecting candidate compounds that selectively bind to CB2;
(b) selecting a compound that is a CB2 inverse agonist among the compounds selected in step (a); and
(c) determining the anti-allergic activity of the compound selected in step (b).

14. A method for treating a non-immediate-type allergic disease, which comprises administering a preparation containing an effective amount of CB2 inverse agonist to a patient affected with the non-immediate-type allergic disease.

15. The method for treating a non-immediate-type allergic disease according to claim 14, wherein the inverse agonist is a compound that exhibits the inverse agonistic action by antagonizing the agonistic action of 2-AG and/or 2-AG-E.

16. The method for treating a non-immediate-type allergic disease according to claim 14, wherein the inverse agonist is a compound selected from the group consisting of: compound A, compound B, compound C, compound D, compound E, compound F, compound G, compound H, compound I and SR144528, and pharmaceutically acceptable salts thereof:

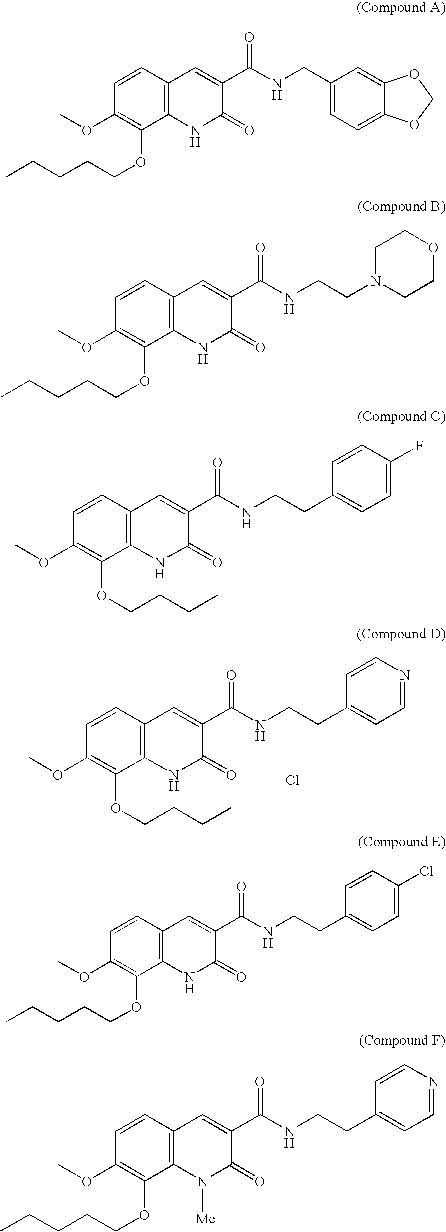

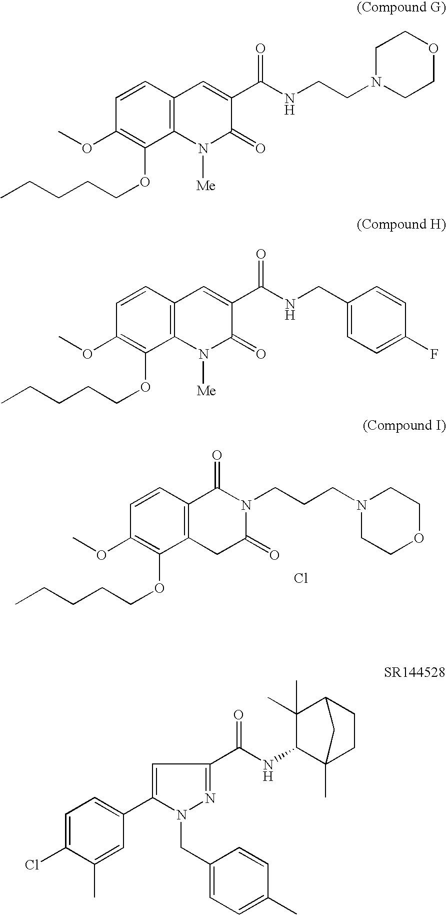

17. The method for treating a non-immediate-type allergic disease according to claim 14, wherein the non-immediate-type allergic disease is selected from the group consisting of: allergic dermatitis, allergic asthma, allergic rhinitis and/or allergic conjunctivitis.

18. A therapeutic agent for a disease associated with 2-AG and/or 2-AG-E, which comprises as an active ingredient an inverse agonist of the peripheral cell type cannabinoid receptor.

19. The therapeutic agent according to claim 18, wherein the disease associated with 2-AG and/or 2-AG-E is selected from the group consisting of: hematologic malignancies, sepsis and diseases of circulatory system.
